(19) United States
(12) Patent Application Publication
(10) Pub. No.: US 2024/0052307 A1
(43) Pub. Date: Feb. 15, 2024

(54) BIOACTIVE COCHLEAR IMPLANT AND APPLICATIONS OF SAME

(71) Applicants: NORTHWESTERN UNIVERSITY, Evanston, IL (US); Cell Guidance Systems Limited, Cambridge (GB)

(72) Inventors: Akihiro J. Matsuoka, Chicago, IL (US); Kevin Tomy Nella, Glenview, IL (US); Michael Jones, Cambridge (GB)

(21) Appl. No.: 18/229,273

(22) Filed: Aug. 2, 2023

Related U.S. Application Data
(60) Provisional application No. 63/398,011, filed on Aug. 15, 2022.

(51) Int. Cl. C12N 5/0793; A61N 1/05; C12N 5/074; C12N 13/00

(52) U.S. Cl. CPC C12N 5/062; A61N 1/0541; C12N 5/0607; C12N 13/00; C12N 2501/13

(57) ABSTRACT

In one aspect, this invention relates to a bioactive implant comprising an electrode array; and a source of neurotrophins coupled with the electrode array for generating a neurotrophin concentration gradient that facilitates a neuro-regenerative nexus (NRN) for survival, neuronal differentiation toward spiral ganglion neurons (SGNs), and directed neurite extension of human pluripotent stem cell (hPSC)-derived SGNs. The invention in another aspect also relates to a method for realization of the NRN in conjunction with an implant, comprising coupling a source of neurotrophins with the electrode array to generate a neurotrophin concentration gradient that facilitates the NRN for survival, neuronal differentiation toward SGNs, and directed neurite extension of hPSC-derived SGNs.

### CROSS-REFERENCE TO RELATED PATENT APPLICATION

This application claims priority to and the benefit of U.S. Provisional Application No. 63/398,011, filed Aug. 15, 2022, which is incorporated herein in its entirety by reference.

### STATEMENT AS TO RIGHTS UNDER FEDERALLY-SPONSORED RESEARCH

This invention was made with government support under grant number W81XWH1810752 awarded by the Department of Defense. The government has certain rights in the invention.

### FIELD OF THE INVENTION

The present invention relates generally to bioengineering, and more particularly to a bioactive cochlear implant for realization of a neuro-regenerative nexus that bridges the electrode-neuron gap and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention. The cochlear implant (CI), which provides functional restoration in patients with sensorineural hearing loss, forms a neuro-electronic interface with the auditory nervous system. CI technology functions by electrically stimulating the extant population of auditory neurons, i.e., spiral ganglion neurons (SGNs). Although cochlear implant (CI) technology has allowed for the partial restoration of hearing over the last few decades, persistent challenges (e.g., poor performance in noisy environments and limited ability to decode intonation and music) remain.

Central to many of these challenges, the “electrode-neuron gap” poses the most significant obstacle to advancing past the current plateau in CI performance. The gap exists between CI electrodes and target membranes of dendrites of surviving endogenous SGNs. It results in the requirement of larger CI excitation fields, which lead to current spread that excites and therefore disables neighboring electrodes, resulting in fewer information channels to the brain. This develops into a vicious cycle as fewer information channels to the brain require larger CI excitation fields. The width of the gap generally spans hundreds of μm. It is demonstrated in vitro that energy needed to elicit a response can be reduced by up to 20% by decreasing the width from 40 to zero μm (early postnatal mouse SGN explants were grown on a microelectrode array).

Previous work has introduced the concept of a “bioactive” CI to resolve the electrode-neuron gap in vivo. The bioactive CI combines current state-of-the-art CI technology with emerging stem cell replacement therapy in the inner ear. In this scheme, transplanted human pluripotent stem cell (hPSC)-derived SGNs bridge the gap between the CI and surviving endogenous SGNs. However, the issue of establishing synaptic connections between the two cell populations remain.

Neurotrophin gradients have been shown to guide hPSC grafts in spinal cord injury, direct growth of endogenous SGNs toward CI electrodes in the scala tympani, and enable transplanted hPSC-derived otic neuronal progenitors (ONPs) to grow neurites toward the modiolus where the cell bodies of SGNs are housed. Although promising, these studies failed to demonstrate sufficient directed neurite outgrowth, e.g., lack of synaptic connections between hPSC grafts and endogenous SGNs, presumably precluding significant improvements in functional recovery of hearing.

Therefore, a heretofore unaddressed need exists in the art to address the aforementioned deficiencies and inadequacies.

### SUMMARY OF THE INVENTION

In one aspect, this invention relates to a bioactive implant to be implanted into a target region of a subject, comprising an electrode array; and a source of neurotrophins coupled with the electrode array for generating a neurotrophin concentration gradient that facilitates a neuro-regenerative nexus (NRN) for survival, neuronal differentiation toward spiral ganglion neurons (SGNs), and directed neurite extension of human pluripotent stem cell (hPSC)-derived SGNs.

In one embodiment, the neurotrophin concentration gradient confers directional neurite growth from transplanted cells in the target region of the subject.

In one embodiment, the neurotrophin concentration gradient comprises a brain-derived neurotrophic factor (BDNF) concentration gradient.

In one embodiment, the NRN is a biological interface that doubly preserves endogenous SGNs while precisely directing growth of neurites arising from transplanted hPSC-derived otic neuronal progenitors (ONPs) toward endogenous SGNs, and vice versa.

In one embodiment, the NRN acts as a supportive bridge between extant SGNs and transplanted hPSC-derived SGNs that are localized on the electrode array.

In one embodiment, the NRN stimulates directed neurite outgrowth from both the hPSC-derived ONPs and the endogenous SGNs via the neurotrophic factor gradient.

In one embodiment, the neurotrophic factor gradient promotes directed neurite growth of the hPSC-derived SGNs and induce synaptogenesis between two such cell populations.

In one embodiment, the NRN integrates the source of neurotrophins with the electrode array to facilitate and maintain the neurotrophic factor gradient.

In one embodiment, a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

In one embodiment, the polyhedrin delivery system comprises a crystalline growth factor formulation to facilitate an extended release of growth factors including neurotrophins.

In one embodiment, the polyhedrin delivery system is configured to encase the growth factors into polyhedrin protein crystals to produce growth factor co-crystals that have slow degradation profiles under physiological conditions, thereby allowing the extended release of the embedded bioactive growth factors.

In one embodiment, the polyhedrin delivery system contains polyhedrin protein and cargo protein co-expressed within the polyhedrin crystal, wherein the cargo protein comprises rhBDNF and is controllably releasable.

In one embodiment, the source of neurotrophins comprises recombinant human brain-derived neurotrophic factors (rhBDNF).

In one embodiment, coupling of the polyhedrin delivery system with the electrode array establishes a neuronal network between transplanted hPSC-derived ONP grafts and extant SGNs in the target region of the subject.

In one embodiment, the establishment of the neural network results in lower electrical impedance and current requirements of the bioactive implant.

The bioactive implant of claim 1, wherein the neuro-regenerative nexus congruent with the bioactive implant eliminates an electrode-neuron gap.

In one embodiment, the bioactive implant is a cochlear implant (CI) implanted into an inner ear of the subject.

In one embodiment, the electrode array is coated with Poly-D-Lysine/laminin with hPSC-derived SGNs.

In one embodiment, the source of neurotrophins is incorporated into a strip or rod that is placed in conjunction with the electrode array to facilitate neural integration between the electrode array and transplanted SGNs and guide neurite outgrowth from native SGNs.

In one embodiment, the strip or rod is formed of a biodegradable and biocompatible thermoplastic polymer, and/or a biocompatible hydrogel.

In another aspect, the invention relates to a method for realization of a neuro-regenerative nexus (NRN) in a target region of a subject. The method comprises placing a bioactive implant into the target region, wherein the bioactive implant comprises an electrode array and a source of neurotrophins; and coupling the source of neurotrophins with the electrode array to generate a neurotrophin concentration gradient that facilitates the NRN for survival, neuronal differentiation toward spiral ganglion neurons (SGNs), and directed neurite extension of human pluripotent stem cell (hPSC)-derived SGNs.

In one embodiment, the neurotrophin concentration gradient confers directional neurite growth from the transplanted cells in the target region of the subject.

In one embodiment, the neurotrophin concentration gradient comprises a brain-derived neurotrophic factor (BDNF) concentration gradient.

In one embodiment, the NRN is a biological interface that doubly preserves endogenous SGNs while precisely directing the growth of neurites arising from transplanted hPSC-derived otic neuronal progenitors (ONPs) toward endogenous SGNs, and vice versa.

In one embodiment, the NRN acts as a supportive bridge between extant SGNs and transplanted hPSC-derived SGNs that are localized on the electrode array.

In one embodiment, the NRN stimulates directed neurite outgrowth from both hPSC-derived ONPs and endogenous SGNs via the neurotrophic factor gradient.

In one embodiment, a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

In one embodiment, the polyhedrin delivery system is configured to encase the growth factors into polyhedrin protein crystals to produce growth factor co-crystals that have slow degradation profiles under physiological conditions, thereby allowing the extended release of the embedded bioactive growth factors.

In one embodiment, the polyhedrin delivery system contains polyhedrin protein and cargo protein co-expressed within the polyhedrin crystal, wherein the cargo protein comprises rhBDNF and is controllably releasable.

In one embodiment, the source of neurotrophins comprises recombinant human brain-derived neurotrophic factors (rhBDNF).

In one embodiment, said coupling of the polyhedrin delivery system with the electrode array establishes a neuronal network between transplanted hPSC-derived ONP grafts and extant SGNs in the target region of the subject.

In one embodiment, said coupling of the polyhedrin delivery system with the electrode array comprises applying current to the electrode array to generate electrical stimulation to the target region; and releasing the embedded bioactive growth factors into the target region.

In one embodiment, the bioactive implant is a cochlear implant (CI) implanted into an inner ear of the subject.

In one embodiment, the electrode array is coated with Poly-D-Lysine/laminin with hPSC-derived SGNs.

In one embodiment, the source of neurotrophins is incorporated into a strip or rod that is placed in conjunction with the electrode array to facilitate neural integration between the electrode array and transplanted SGNs and guide neurite outgrowth from native SGNs.

These and other aspects of the present invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows a next-generation bioactive CI and the neuro-regenerative nexus and polyhedrin delivery system (PODS)® according to embodiments of the invention. Panel A: A next-generation bioactive CI and the neuro-regenerative nexus. The neural network in this scheme consists of a CI electrode (gray), transplanted stem cell-derived SGNs (blue), a neuro-regenerative nexus (orange), and extant/endogenous SGNs (red). Note that neuronal connection is largely absent with hair cells (not shown). Panel B: Polyhedrin Delivery System (PODS)®. PODS® crystals are cubic protein co-crystals produced by Sf9 cells (a clonal isolate of Spodoptera frugaperda Sf21 cells [IPLB-Sf21-AE], in which the polyhedrin protein and a second “cargo” protein are co-expressed) (FIG. 1B, left). S9 cells are commonly used in insect cell culture for recombinant protein production using baculovirus. Inside the Sf9 cells, polyhedrin proteins self-assemble into cubic crystals (panel B of FIG. 1, center) while the active protein, tagged with a short peptide sequence, bind to the growing polyhedrin crystal. The crystals are extracted and purified by simple cell lysis followed by washes to remove cell debris (Scanning electron micrograph, panel B of FIG. 1, right).

FIG. 2 shows an overview of otic neuronal differentiation protocol for hPSCs. Each developmental stage is shown at the top, with key treatments shown below. NNE: nonneuronal ectoderm; PPE: preplacodal ectoderm; EONP: early-stage otic neuronal progenitor; MONP: mid-stage otic neuronal progenitor; LONP: late-stage otic neuronal progenitor; BMP4: bone morphogenetic protein 4; SHH: Sonic hedgehog; ATRA: all-trans retinoic acid; EGF: epidermal growth factor; IGF-1: insulin-like growth factor-1; FGF2: fibroblast growth factor 2; N2B27-CDM: chemically defined medium containing N2 and B27 supplements. LDN193189: a selective BMP receptor inhibitor; SB431542: an inhibitor of the activin receptor-like kinase (ALK) receptors; IWP-2: a potent inhibitor of Wnt processing and secretion; CHIR99021: an aminopyrimidine derivative that is a potent glycogen synthase kinase (GSK) 3 inhibitor; Stem t BasicO2®: undifferentiated hPSC culture media (Ajinomoto Co., Inc., Tokyo, Japan); AS404®: StemFit® For Differentiation (Ajinomoto Co., Inc., Tokyo, Japan).

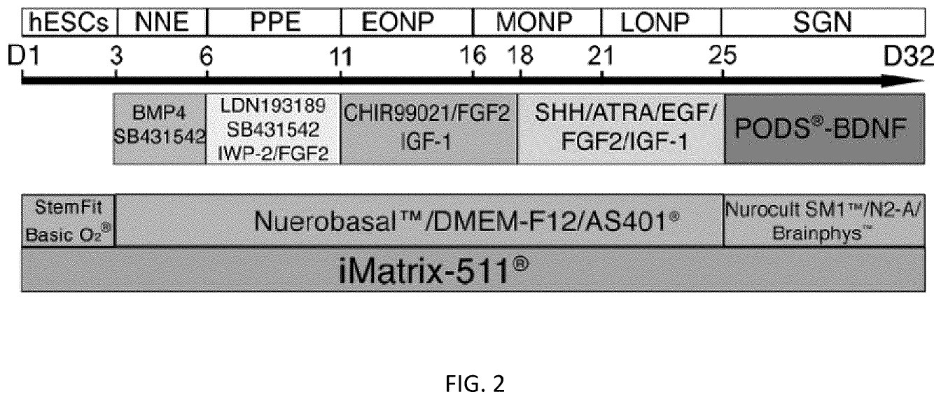

FIG. 3 shows a schematic representation of a Xona™ Microfluidics XC 450 according to embodiments of the invention. Panel A: Schematic representation of a Xona™ Microfluidics XC 450. Two main compartments (somal compartment (SC) and neurotrophin compartment (NTC)) on either side are connected by a micro-groove channel array (MGC) spanning 450 μm, each individual channel having a width of 10 μm (black square). The somal compartment contains hPSC-derived late-stage ONPs (shown in black), while the neurotrophin compartment largely contains the secreted factors (i.e., BDNF) that are released from the PODS®-rhBDNF crystals deposited in its upper well. Panel B: Relevant anatomy of the inner ear associated with a Xona™ XC450 device. SV: scala vestibuli; SM: scala media; ST: scala tympani; SGNs: spiral ganglion neurons; OSL: osseous spiral lamina; CPS: Canaliculae Perforantes of Schuknecht. Scale bar: 200 μm. Panel C: (a) Xona™ Microfluidics XC450 device geometry, created at a 1:1 scale with Autodesk Inventor®, in which the diffusion profile of the released BNDF was modeled. (b) Detail of the microchannels adjoining the two compartments of the Xona™ Microfluidics XC450. (c) Xona™ Microfluidics XC450 device showing the optimal location and geometry of the volume of PODS®-rhBDNF (yellow ellipse). (d): Mesh geometry of the Xona™ XC450 generated with Autodesk Inventor®, depicting the localized volume of PODS®-rhBDNF (1 μL) as an ellipsoid disc. Panel B is reproduced with permission from John Wiley & Sons, Inc., Human cochlea: Anatomical characteristics and their relevance for cochlear implantation. Anatomical Record. 295 (11) (2012) 1791-1811.

FIG. 4 shows chemical kinetics of brain derived neurotrophic factor (BDNF). Panel A: Concentration of rhBDNF in solution over 72 hours after initial application. Panel B: 1/|BDNF|data fit to first order curve. Please refer to other curve fit data in FIG. 13. Red line: fitted line, blue dots: individual data point. k₂ is defined as slope of fitted curve.

FIG. 5 shows analysis of PODS®-rhBDNF according to embodiments of the invention. Panel A: SDS-PAGE analysis of PODS®-rhBDNF. Samples containing six quantities of PODS®-rhBDNF crystals were run on precast 4-15% polyacrylamide mini-gels and stained with Coomassie G-250 solution for visualization. Protein bands appear at approximately 18.8 (a), 28.0 (b), and 46.8 (c) kDa. Lane 1: molecular weight marker. Lanes 2-7: samples containing 1.5×10⁵, 2.0×10⁵, 2.5×10⁵, 3.0×10⁵, 4.0×10⁵, and 5.0×10⁵ PODS®-rhBDNF crystals. Panel B: Western blot analysis of PODS®-rhBDNF crystals. Samples containing six quantities of PODS®-rhBDNF crystals were run on SDS-PAGE and blotted with BDNF polyclonal antibody. Protein bands appear at approximately 14.0 (a), 18.8 (b), 37.6 (c), and 46.8 (d) kDa. Lane 1: molecular weight marker. Lanes 2-7: samples containing 1.5×10⁵, 2.0×10⁵, 2.5×10⁵, 3.0×10⁵, 4.0×10⁵, and 5.0×10⁵ PODS®-rhBDNF crystals.

FIG. 6 shows 3D mesh geometry of a Xona™ XC450. Panel A: 3D mesh geometry of a Xona™ XC450 created with Autodesk Inventor®. A PODS®-rhBDNF ellipsoid disc is shown in yellow. Panel B: rhBDNF concentration gradient for 20,000 PODS®-rhBDNF from D1-D7. Note that the model does not account for media changes during the culture period. The corresponding color map shown has a range from 0 ng/mL to 49 pg/mL. Panel C: Diffusion flux (rnol/m 2 s) was computed using COMSOL Chemical Engineering module on D1, D3, D5, and D7.

FIG. 7 shows human PSC-derived late-stage ONPs grown in a microfluidic device at Day 7 were stained for GATA3 (red) and PAX8 (green). Panel A: Mesh geometry of the microfluidic device Xona™ XC450 constructed with Autodesk Inventor. The green square indicates the cell seeding location in the somal compartment of the device. An ellipsoid PODS®-rhBDNF disc is shown in yellow. Panel B: Cells were analyzed by staining with neuronal markers GATA3 (red) and PAX8 (green). Yellow highlighted regions are magnified on the left. SC: somal compartment. Panel C: Heat-map representation of double-positivity of hPSC-derived ONPs in the device for GATA3 and PAX8 (n=3). Double-positivity increases from the lower to the upper portion of the somal compartment, suggesting that the higher BDNF concentration in the upper portion of the device improves differentiation. Scale bar: 100 μm. Panel D: Prediction of BDNF concentration gradient released from PODS®-rhBDNF at seven days in culture using the finite element model. The corresponding color map is shown with a range from 0-49 pg/mL.

FIG. 8 shows the Euclidean distance angles and diffusion flux anges according to embodiments of the invention. Panel A: Defining the Euclidean distance angle (EDA). (a) X-Y plane mesh geometry of a Xona™ XC450 device. Green square shows the area corresponding to the phase-contrast image below. An ellipsoid PODS®-rM3DNF disc is shown in yellow. (b) Yellow ellipse indicates the location of a disc containing PODS®-rhEIDNF crystals (P: the center of this ellipse). In the somal compartment of the Xona™ XC450 device, we defined Zone 1-5, shown in black squares. A line was drawn from the center of the PODS®-rhBDNF disc (P) to (Q₁₋₅) (i.e., the side nearest to microgroove channels) of a pre-designated square (shown as a black square, zone 1-5) [i.e., zone 1-5]). The Euclidean distance angle (EDA) for zone 1-5 was defined as φᵢ=1-5. Note that phase-contrast microscopy does not allow for clear visualization of cells in the microgroove channels due to the refraction pattern within the Xona™ XC450 device. Panel B: Defining diffusion flux angle (DFA). (a) Diffusion flux in the Xona™ XC450. Green squared area shows somal and neurotrophin compartments, which are magnified in (b). (b) Magnified image of diffusion flux in zones 1-5 in the Xona™ XC450. Orange highlighted zone (zone 4) was magnified at the bottom of (b), defining the DFA (ψ).

FIG. 9 shows preferred cell orientation analysis according to embodiments of the invention. Sequential phase-contrast images of the somal compartment of the Xona™ XC450 device in zones 1-5 (see FIG. 8) on day 1 (D1), day 3 (D3), and day 7 (D7). A corresponding polar histogram indicates preferred orientation of hPSC-derived ONPs as an angle (0-2w radians) in blue circles. Mean vector angle is shown in red line. Green dotted line shows Euclidean Distance Angle (EDA) and purple triangle indicates Diffusion Flow Angle (DFA). t symbol indicates that p value >0.01. * symbol indicates that the mean vector angle fits within the 95% confidence internal (α<0.05.)

FIG. 10 shows immunocytochemistry and angles of hPSC-derived ONPs cultured in the Xona™ XC450 according to embodiments of the invention. Panel A: Immunocytochemistry of hPSC-derived ONPs cultured in the Xona™ XC450 for seven days with /3-III-Tubulin (pink fluorescence) and DAPI (blue fluorescence). A green line was drawn from the center of the BDNF disc (P) to the mid point of each of three pre-determined squares (Regions 1-3) in the somal compartment to define Euclidean Dis-tance Angle (EDA: φ′ᵢi=1-3). Green square: somal compartment (SC); yellow square: microgroove channels (MCC); white square: Neurotrophin compartment (NTC). High-pow magnified images of the NTC and SC are shown. An ellipsoid PODS®-rhBDNF disc is shown in yellow. P: the center of the disc. Panel B: Polar histogram of neurite directional angles of hPSC-derived ONPs in the Xona™ XC450 at Day 7 (D7). The two longest neurites were measured. EDA: green dotted line. DFA: purple triangle. Panel C: (a) Right: Polar histogram of neurite directional angles of hPSC-derived ONPs cultured with 20 ng/mL of recombinant human BDNF for seven days. Left: Corresponding photomicrograph of immunocytochemistry with β-III tubulin (pink) and DAPI (blue). (b) Right: Polar histogram of neurite directional angles of hPSC-derived ONPs cultured with 800,000 of PODS®-rhBDNF for seven days. Left: Corresponding photomicrograph of immunocytochemistry with f-III tubulin (pink) and DAPI (blue). Panel D: A circular plot of neurite directional angle of hPSC-derived ONPs in Region 1-3. Once again, the two longest neurites were plotted on a unit circle. Right column: Original directional angle distribution. Left column: the method of doubling the angle applied to the original data. Each datum point is shown as a blue circle. Mean vector angle is shown as a red line. Green dotted line shows Euclidean Distance Angle (EDA) and purple triangle indicates Diffusion Flow Angle (DFA). * symbol indicates that the mean vector angle fits within the 95% confidence internal (a<0.05.)

FIG. 11 shows human PSC-derived late-stage ONPs in the microfluidic device were stained with β-III tubulin (red) and DAPI (blue) to measure cell migration. Panel A: Late-stage ONPs stained after seven days in culture (a) with regions highlighted and magnified in the somal compartment (b), microgroove channels (c), and neurotrophin compartment (d). SC: somal compartment, MGC: microgroove channels, NTC: neurotrophic compartment. An ellipse-shaped PODS®-rhBDNF disc is shown in yellow. Scale bar (a): 500 μm; (b)-(d): 100 μm. Panel B: Quantitative analyses of cell migration and neurite extension in devices using recombinant human BDNF, 800,000 PODS®-rhBDNF, or 20,000 PODS®-rhBDNF. *: p<0.05, **: p<0.01, ***: p<0.001.

FIG. 12 shows immunocytochemistry of hPSC-derived SGNs and RFP-positive hPSC-derived SGN clones cultured in Xona™ XC450 devices for panel A: seven days in vitro (DIV7), and panel B: three days in vitro (DIV3) (control) with -III-tubulin (green uorescence), RFP (red uorescence), and synapsin 1/2 (blue uorescence). Synapsin 1/2 expression was observed between distinct hPSC-derived SGN populations in the microgroove channels at DIV7 (A; red and yellow squares), but not at DIV3 (B; yellow square). Note that images are pseudo-colored. (C): Quanti cation of the number of synaptic puncta in the microgroove channels at DIV7 and DIV3. n=3 (three technical replicates [each eld contained 10 microgroove channels×3)] with three biological replicates [three XC450 devices]). **: p<0.01. SC: somal compartment; MGC: microgroove channels; NTC: neurotrophin compartment; RFP: red-uorescent protein; BIIIT: β-III tubulin; SNP: synapsin 1/2; DIV: day in vitro in-vitro; Triangled-arrowhead: synapsin 1/2 positive; White arrow: RFP-positive hPSC-derived SGNs (SC); White double arrow: β-III tubulin-positive hPSC-derived SGNs (NTC). Scale bar: 100 μm.

FIG. 13 shows fluorescence images for a secondary antibody control experiment using donkey anti-goat Alexa 488 (D-anti-Gt 488: blue channel, panel A), donkey anti-mouse (D-anti-Ms-594: Green, panel B), donkey anti-rabit Alexa 647 (D-anti-Rb-647: Red, panel C), and TOTO-3 Iodide (counterstain: Far-red, panel D). Each sample was treated identically to experimental inununocytochemistry conditions with the omission of primary antibody during overnight incubation. TOTO-3 Iodide image was taken followed by a secondary antibody only condition. D: donkey, G: goat, Ms: mouse, and Rb: rabbit. Cells are all hESC-derived ONPs (P5) cultured on a cover glass coated with iMatrix-511 laminin solution and visualized at 10× on Nikon Ti2 Widefield microscope. Each channel was viewed on 1 sec exposure.

FIG. 14 shows automated image-processing strategies (panels A-E) and measurement of cell orientation angles according to embodiments of the invention. See detailed description in the text.

FIG. 15 shows polar coordinate system and images of hESC-derived ONPs according to embodiments of the invention. Panel A: Polar coordinate system to determine the directionality. Red line indicates the direction and magnitude of the mean vector. X and Y-axis are shown in green dotted line. Panel B: Right, a binarized image of hESC-derived ONPs, and Left, a magnified image. Panel C: further magnified image of an hESC-derived ONE. Panel D: A cell example we used for preferred cell orientation analysis. The direction and length of the ellipse's axes are given by the eigenvectors and eigenvalues of the covariance matrix. This figure illustrates the axes (the longer red line) and orientation of the ellipse that represents cell orientation. The red lines show the axes (major and minor axis) and the gray dots are showing the foci. The cell orientation angle was defined as the angle between the horizontal line (dark blue line) and the major axis (the longer red line) denoted by B. Please also see details in the text below.

FIG. 16 shows images and angle analysis of hESC-derived ONPs according to embodiments of the invention. Panel A: An eight-bit, gray-scaled image of hESC-derived ONPs, which were cultured in a Xona™ XC450 for seven days. They were immunostained with tubulin and DAPI. A typical bipolar neuron is shown here. Panel B: An image shown in A is an inverse gray scale image to show the axes to define θ (neurite #1) and θ′ (neurite #2). Panel C: A polar plot indicates the bipolar nature of hESC-derived ONPs (blue circles). We assumed that the distribution of our samples is bimodal. As a result, the mean vector, r, would contain little information (red line representing the mean vector r). Panel D: After applying the method of doubling the angles (each angle was doubled so as to reduce the multiple modulo 360 degrees). The manipulation resulted in unimodal circular distribution. The biomodal directions are now clustered around unidirectional distribution: resulting in more meaningful mean vector r.

FIG. 17 shows a polar coordinate plotting 30 observed angle θ₁₋₃₀ (blue circles) obtained from preferred cell orientation analysis, according to embodiments of the invention. Note that each of the corresponding unit vector as 1. Green-dotted lines: X and Y-axis.

FIG. 18 shows different views according to embodiments of the invention. Panel A: A view on a X-Y plane. Panel B: A view on a X-Z plane. Note that thick-ness/height of a soma channel and a neurotrophin channels are both 100 μm and that of a microgroove channel is 3 μm. Panel C: A view on a 3-D oblique view.

FIG. 19 shows kinetic data according to embodiments of the invention. Panel A: Zero-order kinetics. Panel B: First-order kinetics. Panel C: Second-order kinetics. Panel D: An explanatory screenshot of non-linear curve fitting procedure using MATLAB Curve Fitting Toolbox. One of the most common tasks in chemical reaction kinetics is to extract the rate constant from the time dependence of chemical concentration such as BDNF concentration. We considered three degrees of orders—a zero order, a first order, and a second order in this study. We performed a linear (zero order) and nonlinear (first and second order) least square analysis of kinetic data obtained from ELISA by using MATLAB Curve Fitting Toolbox (Mathworks, Natick, MA, USA). FIG. 19 shows the curve fitting data (panels A-C). FIG. 19 (panel D) indicated a typical analysis using MATLAB Curve Fitting Toolbox. We found that highest corresponding R² was 0.97972 for the first order kinetics, confirming that the degradation is indeed the first order kinetics.

FIG. 20 shows a finite element model with 10,000 PODS-BDNF (upper set) and 40,000 PODS-BDNF (lower set) from Day 1-7, according to embodiments of the invention. We have included a few different hypothetical numbers of PODS-BDNF for our FEM to optimize the BDNF concentration gradient in a Xona XC450. Briefly, we first used 10,000 PODS-BDNF, however, the number was clearly not sufficient to establish a BDNF concentration gradient at 3-5 days to induce suitable otic neuronal differentiation and neurite outgrowth at day 7. We then used 40,000 PODS-BDNF, with which the BDNF concentration gradient was uniformly distributed throughout DIV 1-7, which apparently lacking BDNF gradient. The number of PODS 20,000 was chosen because 1) this condition allowed for generating BDNF concentration gradient that was sustained at the optimal time (i.e., 3-5 days) and also high enough (i.e., around 50 pg/mL) to promote otic neuronal differentiation as well as neurite extension of hESC-derived ONPs. Note that our previous ELISA showed a steady rate of release of BDNF from PODS-BDNF crystals at approximately 25-50 pg/mL/day, which promoted otic neuronal differentiation of hESC-derived ONPs as well as enhanced axonal growth from them. Please see our FEM data with 10,000 of PODS and 40,000 of PODS in FIG. 20.

FIG. 21 shows an explanatory Q-Q plot for preferred cell orientation angles in Region 1 on day 1, according to embodiments of the invention.

FIG. 22 shows immunocytochemistry of hESC-derived ONPs cultured in a Xona™ XC450 for seven days with (panel a) PODS-BDNF and (panel b) recombinant human BDNF, according to embodiments of the invention. The photomicrographs were taken in a soma channel. Yellow dotted line was drawn from the PODS-BDNF disk to a hESC-derived SGN, which extended the greatest number of neurites in each figure (yellow empty circle). Yellow filled circle (P) indicates the location of PODS-BDNF.

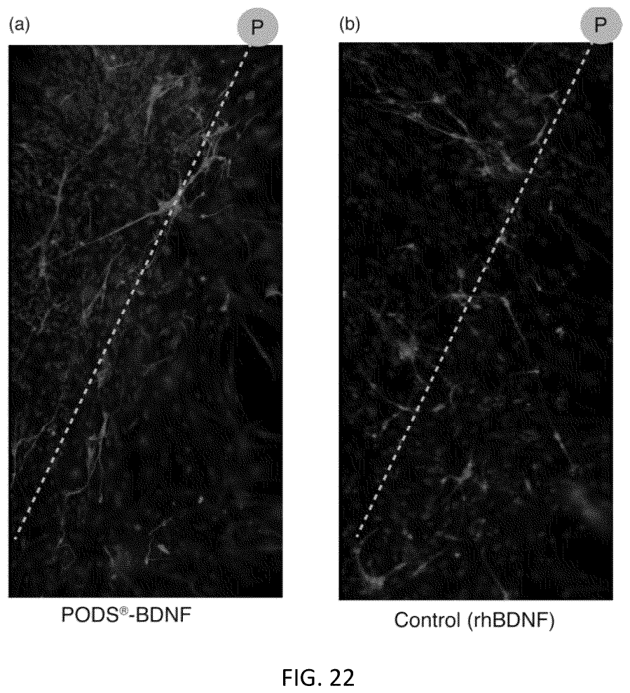

FIG. 23 shows electrophoresis buffer for sample condition and run condition according to embodiments of the invention.

FIG. 24 shows the Euclidean distance angle and diffusion flux angle, descriptive statistics (mean vector angle, median vector angle, angular variance, angular standard deviation), and inferential statistics (the Reyleigh test, the V test), and test concerning means and median (One sample test for the mean angle) in zone 1-5 on Day 1, 3, and 5, according to embodiments of the invention. Note that the V-test statistics in Zone 5 was not statistically significant (green highlighted). Also note the yellow-highlighted cells indicating that they are statistically significant on one sample test for the mean angle.

FIG. 25 shows the Euclidean distance angle and diffusion flux angle, descriptive statistics (mean vector angle, median vector angle, angular variance, angular standard deviation), and inferential statistics (the Reyleigh test, the V test), and test concerning means and median (One sample test for the mean angle) in region 1-3 on Day 1, 3, and 5, according to embodiments of the invention. Note that the Reyleigh test statistics in Zone 5 was not statistically significant (green highlighted).

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

Although cochlear implant (CI) technology has allowed the patient population to partially restore the sense of hearing over the last few decades, persistent challenges remain, including the deciphering of a rich acoustic signal into an electrical pulse-train signal. Among these challenges, the “electrode-neuron gap” poses a significant obstacle to advancing past the current plateau in CI performance, resulting in limited performance in a noisy background and a poor ability to decode intonation and music. We propose the development of a “neurotrophic strip”-biological interface that doubly preserves endogenous spiral ganglion neurons (SGNs) while precisely directing the growth of neurites arising from transplanted human pluripotent stem cell (hPSC)-derived otic neuronal progenitors (ONPs), toward endogenous SGNs. We hypothesized that Polyhedrin Delivery System PODS®-human brain-derived neurotrophic factor (BDNF) could stably provide an adequate BDNF gradient to hPSC-derived ONPs, thereby facilitating otic neuronal differentiation and directional neurite outgrowth. To test this hypothesis, we first utilized a finite element model to simulate the in vitro BDNF gradient generated by PODS. For biological verification, we validated the concept of the neurotrophic strip by using a multi-chamber microfluidic device, which mimics the in vivo micro-environment more so than conventional laboratory plates in terms of volume and concentrations of endogenous/exogenous factors. We were able to generate BDNF concentration gradient, enabling survival, neuronal differentiation toward hPSC-derived SGNs, and directed neurite extension of hPSC-derived SGNs. The technology will allow us to further advance the concept of “neurotrophic strip” further in the inner ear by controlling neurite direction of transplanted hPSC-derived ONPs, providing a step toward next-generation bioacitve CI technology.

In one aspect, this invention relates to a bioactive implant to be implanted into a target region of a subject, comprising an electrode array; and a source of neurotrophins coupled with the electrode array for generating a neurotrophin concentration gradient that facilitates a neuro-regenerative nexus (NRN) for survival, neuronal differentiation toward spiral ganglion neurons (SGNs), and directed neurite extension of human pluripotent stem cell (hPSC)-derived SGNs.

In one embodiment, the neurotrophin concentration gradient confers directional neurite growth from transplanted cells in the target region of the subject.

In one embodiment, the neurotrophin concentration gradient comprises a brain-derived neurotrophic factor (BDNF) concentration gradient.

In one embodiment, the NRN is a biological interface that doubly preserves endogenous SGNs while precisely directing growth of neurites arising from transplanted hPSC-derived otic neuronal progenitors (ONPs) toward endogenous SGNs, and vice versa.

In one embodiment, the NRN acts as a supportive bridge between extant SGNs and transplanted hPSC-derived SGNs that are localized on the electrode array.

In one embodiment, the NRN stimulates directed neurite outgrowth from both the hPSC-derived ONPs and the endogenous SGNs via the neurotrophic factor gradient.

In one embodiment, the neurotrophic factor gradient promotes directed neurite growth of the hPSC-derived SGNs and induce synaptogenesis between two such cell populations.

In one embodiment, the NRN integrates the source of neurotrophins with the electrode array to facilitate and maintain the neurotrophic factor gradient.

In one embodiment, a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

In one embodiment, the polyhedrin delivery system comprises a crystalline growth factor formulation to facilitate an extended release of growth factors including neurotrophins.

In one embodiment, the polyhedrin delivery system is configured to encase the growth factors into polyhedrin protein crystals to produce growth factor co-crystals that have slow degradation profiles under physiological conditions, thereby allowing the extended release of the embedded bioactive growth factors.

In one embodiment, the polyhedrin delivery system contains polyhedrin protein and cargo protein co-expressed within the polyhedrin crystal, wherein the cargo protein comprises rhBDNF and is controllably releasable.

In one embodiment, the source of neurotrophins comprises recombinant human brain-derived neurotrophic factors (rhBDNF).

In one embodiment, coupling of the polyhedrin delivery system with the electrode array establishes a neuronal network between transplanted hPSC-derived ONP grafts and extant SGNs in the target region of the subject.

In one embodiment, the establishment of the neural network results in lower electrical impedance and current requirements of the bioactive implant.

The bioactive implant of claim 1, wherein the neuro-regenerative nexus congruent with the bioactive implant eliminates an electrode-neuron gap.

In one embodiment, the bioactive implant is a cochlear implant (CI) implanted into an inner ear of the subject.

In one embodiment, the electrode array is coated with Poly-D-Lysine/laminin with hPSC-derived SGNs.

In one embodiment, the source of neurotrophins is incorporated into a strip or rod that is placed in conjunction with the electrode array to facilitate neural integration between the electrode array and transplanted SGNs and guide neurite outgrowth from native SGNs.

In one embodiment, the strip or rod is formed of a biodegradable and biocompatible thermoplastic polymer, and/or a biocompatible hydrogel.

In another aspect, the invention relates to a method for realization of a neuro-regenerative nexus (NRN) in a target region of a subject. The method comprises placing a bioactive implant into the target region, wherein the bioactive implant comprises an electrode array and a source of neurotrophins; and coupling the source of neurotrophins with the electrode array to generate a neurotrophin concentration gradient that facilitates the NRN for survival, neuronal differentiation toward spiral ganglion neurons (SGNs), and directed neurite extension of human pluripotent stem cell (hPSC)-derived SGNs.

In one embodiment, the neurotrophin concentration gradient confers directional neurite growth from the transplanted cells in the target region of the subject.

In one embodiment, the neurotrophin concentration gradient comprises a brain-derived neurotrophic factor (BDNF) concentration gradient.

In one embodiment, the NRN is a biological interface that doubly preserves endogenous SGNs while precisely directing the growth of neurites arising from transplanted hPSC-derived otic neuronal progenitors (ONPs) toward endogenous SGNs, and vice versa.

In one embodiment, the NRN acts as a supportive bridge between extant SGNs and transplanted hPSC-derived SGNs that are localized on the electrode array.

In one embodiment, the NRN stimulates directed neurite outgrowth from both hPSC-derived ONPs and endogenous SGNs via the neurotrophic factor gradient.

In one embodiment, a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

In one embodiment, the polyhedrin delivery system is configured to encase the growth factors into polyhedrin protein crystals to produce growth factor co-crystals that have slow degradation profiles under physiological conditions, thereby allowing the extended release of the embedded bioactive growth factors.

In one embodiment, the polyhedrin delivery system contains polyhedrin protein and cargo protein co-expressed within the polyhedrin crystal, wherein the cargo protein comprises rhBDNF and is controllably releasable.

In one embodiment, the source of neurotrophins comprises recombinant human brain-derived neurotrophic factors (rhBDNF).

In one embodiment, said coupling of the polyhedrin delivery system with the electrode array establishes a neuronal network between transplanted hPSC-derived ONP grafts and extant SGNs in the target region of the subject.

In one embodiment, said coupling of the polyhedrin delivery system with the electrode array comprises applying current to the electrode array to generate electrical stimulation to the target region; and releasing the embedded bioactive growth factors into the target region.

In one embodiment, the bioactive implant is a cochlear implant (CI) implanted into an inner ear of the subject.

In one embodiment, the electrode array is coated with Poly-D-Lysine/laminin with hPSC-derived SGNs.

In one embodiment, the source of neurotrophins is incorporated into a strip or rod that is placed in conjunction with the electrode array to facilitate neural integration between the electrode array and transplanted SGNs and guide neurite outgrowth from native SGNs.

Exemplary embodiments demonstrate that the generation of in vitro neurotrophin concentration gradients facilitates survival, neuronal differentiation toward auditory neurons, and directed neurite extension of human pluripotent stem cell-derived auditory neurons. These findings are indispensable to designing a bioactive cochlear implant, in which stem cell-derived neurons are integrated into a cochlear implant electrode strip, as the strategy will confer directional neurite growth from the transplanted cells in the innerear. This study is the first to present the concept of a “neuro-regenerative nexus” congruent with a bioactivecochlear implant to eliminate the electrode-neuron gap—the most significant barrier to next-generation cochlear implant technology.

These and other aspects of the invention are further described below. Without intent to limit the scope of the invention, exemplary instruments, apparatus, methods, and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Mitigating the Electrode-Neuron Gap: Neurotrophic Gradient-Induced Neurite Guidance Through Finite Element Analysis

Although cochlear implant (CI) technology has allowed for the partial restoration of hearing over the last few decades, persistent challenges (e.g., poor performance in noisy environments and limited ability to decode intonation and music) remain. The “electrode-neuron gap” poses the most significant obstacle to advancing past the current plateau in CI performance. Resulting issues include poor performance in noisy environments and a limited ability to decode intonation and music.

To address this issue, we disclose a “neuro-regenerative nexus”—a biological interface that doubly preserves endogenous spiral ganglion neurons (SGNs) while precisely directing the growth of neurites arising from transplanted human pluripotent stem cell (hPSC)-derived otic neuronal progenitors (ONPs) toward endogenous SGNs. We hypothesized that the Polyhedrin Delivery System (PODS®-human brain-derived neurotrophic factor (BDNF)) could stably provide an adequate BDNF concentration gradient to hPSC-derived ONPs and thereby facilitate otic neuronal differentiation and directional neurite outgrowth. To test this hypothesis, a finite element model was constructed in order to simulate the in vitro BDNF concentration gradient generated by the PODS®. For biological validation, cell culture experiments were conducted using a multi-chamber microfluidic device that more closely mirrors the micro-environment of the inner ear than conventional laboratory plates, especially in terms of volume and concentrations of endogenous factors. We were able to generate the optimal BDNF concentration gradient to enable survival, neuronal differentiation toward SGNs, and directed neurite extension of hPSC-derived SGNs. The technique allows us to further advance our concept of a “neuro-regenerative nexus” by controlling neurite direction of transplanted hPSC-derived ONPs and their endogenous counterparts. This proof-of-concept study provides a step toward the next generation of CI technology.

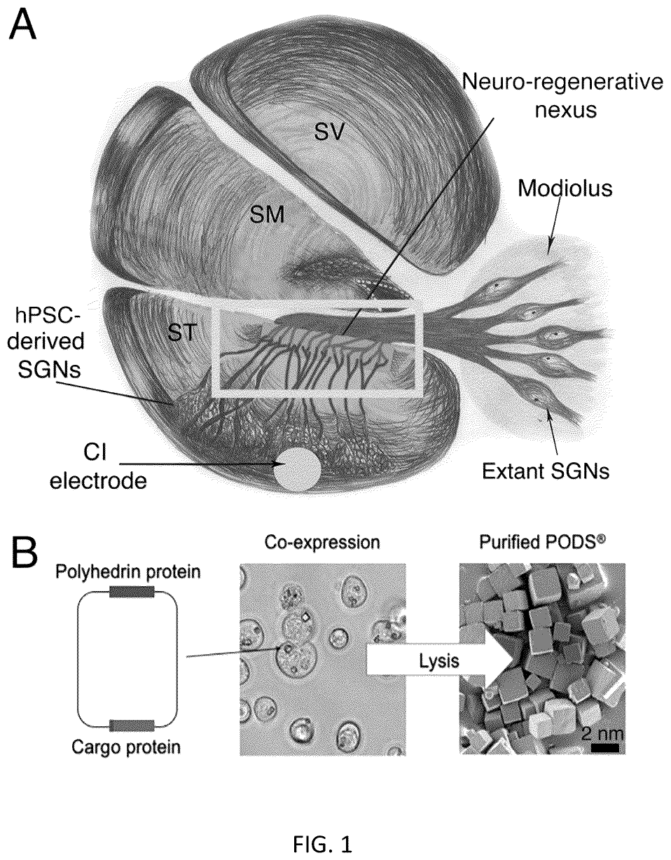

The highlighted yellow-square area in panel A of FIG. 1 shows a schematic diagram of a “neuro-regenerative nexus”—a bioengineered interface that doubly preserves endogenous SGNs and precisely directs the growth of neurites arising from differentiated transplanted hPSC-derived ONPs toward the endogenous SGNs, and vice versa. Here, the neuro-regenerative nexus (shown as an orange rectangle in panel A of FIG. 1) stimulates directed neurite outgrowth from both hPSC-derived ONPs and endogenous SGNs via a neurotrophic factor gradient. While the concept of using neurotrophin gradients for directional axonal growth has existed for a few decades, implementation has been extremely challenging due to the lack of self-sustaining neurotrophin delivery methods. One major contributor is the biochemical instability of neurotrophins, which suffer from structural fragility and thermo-instability under normal physiological conditions both in vitro and in vivo, resulting in half-lives typically ranging from minutes to hours. We set out to mitigate this phenomenon by utilizing the Polyhedrin Delivery System (PODS®), a crystalline growth factor formulation developed to facilitate extended release of growth factors, e.g., neurotrophins (panel B of FIG. 1). The PODS® technology has adapted viral machinery to encase a selected growth factor into polyhedrin protein crystals. The resultant growth factor co-crystals have slow degradation profiles under physiological conditions and, therefore, allow the sustained release of the embedded bioactive growth factors.

We reasoned that a bio-engineered scaffolding integrated with the PODS® technology can establish a neuronal network between transplanted hPSC-derived ONP grafts and extant SGNs in the inner ear. More specifically, we hypothesized that a precisely controlled neurotrophic factor gradient can promote directed neurite growth of human stem cell-derived SGNs and induce synaptogenesis between two such cell populations. When translated in vivo, the formation of a neural network between stem cell-derived SGNs and native SGNs results in lower electrical impedance and current requirements. This phenomenon also leads to less current spread and, as a result, recapitulates the decreased current requirement. These sequelae also results in longer battery life. To test this hypothesis, we constructed a finite element model (FEM) to simulate the in vitro neurotrophin concentration gradient generated by the PODS®. In this study, we focus on BDNF, the most studied of the neurotrophins in the inner ear, and the most vital for functional recovery of damaged SGNs. For biological validation we employed a multi-chamber microfluidic device that more closely mirrors the in vivo micro-environment of the inner ear than conventional laboratory plates in terms of geometry, volume and concentrations of endogenous factors.

### Materials and Methods

Polyhedrin delivery system: The Polyhedrin Delivery System (PODS®-recombinant human BDNF (rhBDNF)) (Cell Guidance Systems, Cambridge, United Kingdom) was used as the self-sustaining source of rhBDNF. PODS®-rhBDNF is composed of the polyhedrin protein formed by Bombyx morn, an insect from the moth family Bombycidae. A cargo protein (i.e., rhBDNF) is co-expressed within the polyhedrin crystal and is slowly released by breakdown of the PODS® crystals via cell-secreted proteases (panel B of FIG. 1).

Enzyme-linked immunosorbent assay for brain-derived neurotrophic factor: Concentrations of rhBDNF were measured at sequential time points to determine the degradation and release kinetics of PODS®-rhBDNF crystals. Culture media from control and experimental conditions were collected at each time point and immediately stored at −80° C. An enzyme-linked immunosorbent assay (ELISA) was performed after the final collection. The same method was applied to measure the degradation kinetics of rhBDNF protein with a carrier protein (Bovine Serum Albumin (BSA)) (#248-BDB-050, R&D Systems, Minneapolis, Minnesota, USA). Experimental conditions were culture media enriched with 10% fetal bovine serum (FBS) (Thermo Fisher Scientific, Waltham, MA, USA). All rhBDNF samples were quantified with a BDNF ELISA kit (#BGK23560; PeproTech, Rocky Hill, New Jersey, USA), and the results were analyzed with a Synergy HTX Multi-Mode Reader (BioTek, Winocski, Vermont, USA) at a 450 nm wavelength, as instructed by the manufacturer. Molecular kinetics were then calculated using a self-coded curve fit analysis as well as the MATLAB Curve Fitting Toolbox (MathWorks, Natick, CA, USA). These kinetics data are consequential in the finite element model, to be described in a later section.

Sodium dodeepi sulphate-polyacrylamide gel electrophoresis: Sodium dodecyl-sulfate polyacrylamide gel electrophoresis (SDS-PAGE) was used to formulate the molar ratio of polyhedrin to BDNF in the PODS®-rhBDNF, required for an exceptionally accurate finite element analysis, described in a later section. SDS-PAGE is commonly used as a method to separate proteins with molecular masses between 5 and 250 kDa, which is suitable for detecting rhBDNF (molecular weight (MW): 14 kDa) and polyhedrin (MW: about 29 kDa). Briefly, each protein sample was diluted in deionized water and mixed with 6× Laemmli sample buffer (Bio-Rad Laboratories, Inc., Des Plaines, Illinois, USA) containing 2-mercaptoethanol and heated at 100° C. for 5 to 20 minutes. Samples were then loaded into precast Mini-PROTEAN TGX 4-15% polyacrylamide mini-gels (Bio-Rad Laboratories, Inc., Des Plaines, IL, USA). Then, 5 mL of Precision Plus Protein Kaleidoscope Prestained Protein Standards (Bio-Rad Laboratories, Inc., Des Plaines, IL, USA) were loaded in each gel run. Electrophoresis was performed at room temperature for approximately 90 minutes using a constant voltage (100V) in 1× solution of Tris-Glycine-SDS electrophoresis buffer (Bio-Rad Laboratories, Inc., Des Plaines, IL, USA) until the dye front reached the end of the 60 mm gel. After electrophoresis, the mini-gels were rinsed with deionized water 3 times for 5 minutes and were subsequently incubated in SimplyBlue™ SafeStain (ThermoFisher Scientific, Waltham, MA, USA) for one hour at room temperature with gentle agitation. Images obtain from gels were analyzed using ImageJ 1.53 g (Dec. 4, 2020, the National Institutes of Health, Bethesda, MD, USA). The calculated molar ratio from the analysis was applied to the COMSOL® Multiphysics model to accurately predict the rhBDNF concentration gradient arising from PODS®-rhBDNF. SDS-PAGE was performed according to the manufacturer's technical guide (Bio-Rad Laboratories, Inc., Des Plaines, IL, USA).

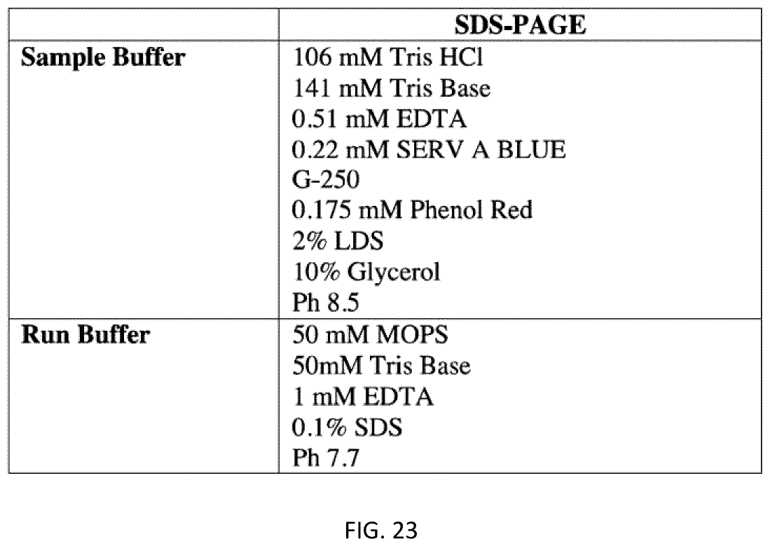

Western Blot: The identity of the rhBDNF protein detected by SD S-PAGE was confirmed by Western blot (Bio-Rad Laboratories, Inc., Des Plaines, IL, USA). The polyvinylidene difluoride (PVDF) membrane was prepared in methanol for 30 seconds before soaking in 1× Tris-Glycine-Methanol transfer buffer for 10 minutes. Wet transfer was performed at 4° C. for approximately 60 minutes using a constant voltage (100 V) in 114 solution of Tris-Glycine-Methanol transfer buffer. After transfer, the membrane was briefly rinsed with 114 Tris-buffered saline Tween-20 (TBST) and was subsequently blocked in 5% BSA in TBST for 24 hours at 4° C. with gentle agitation. The membrane was then rinsed with 1× TBST before incubating with BDNF polyclonal antibody (ThermoFisher Scientific, Waltham, MA, USA) diluted to 1:1500 in 1% BSA in TBST for 24 hours at 4° C. with gentle agitation. Following incubation, the membrane was rinsed in 1× TBST 5 times for 5 minutes to remove unbound primary antibody. Next, the membrane was incubated in Goat anti-Rabbit IgG (H+L) horseradish peroxi-dase (HRP) conjugated secondary antibody (ThermoFisher Scientific, Waltham, MA, USA) diluted to 1:5000 in 1% BSA in TBST for one hour at room temperature with gentle agitation. Following incubation, the membrane was rinsed in 1×TBST 5 times for 5 minutes to remove unbound secondary antibody. For sensitive detection, the membrane was treated with Pierce™ ECL Western Blotting Substrate (ThermoFisher Scientific, Waltham, MA, USA), and visualized using an Azure 600 Digital Imager (Azure Biosystems, Dublin, CA, USA). Electrophoresis buffer for sample condition and run condition was summarized in FIG. 23.

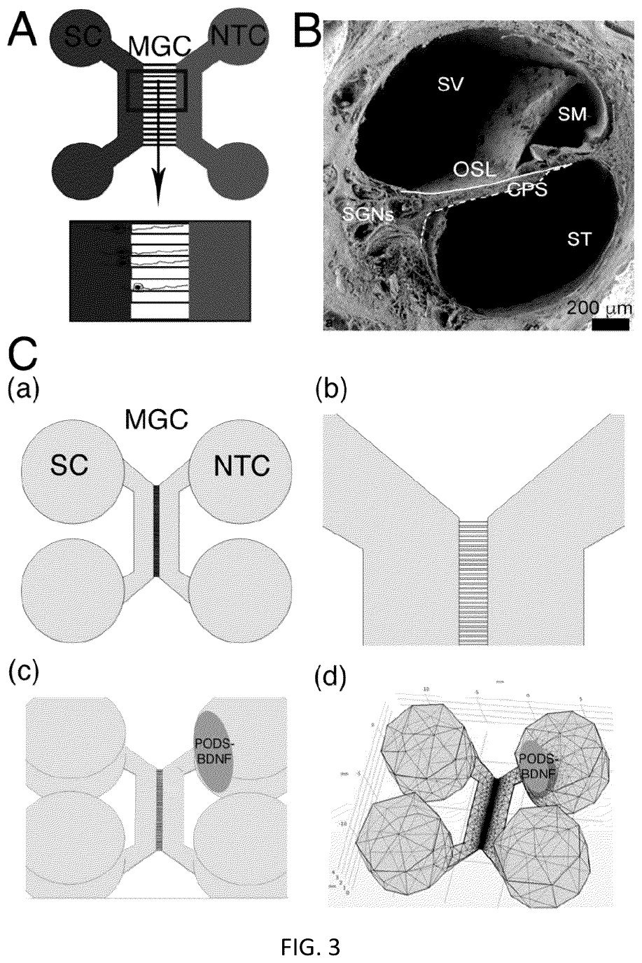

Three-dimensional finite element analysis: We used finite element analysis (FEA) to simulate the BDNF concentration gradient over time in a multi-chamber microfluidic device. FEA is a computational numerical technique, which approximates mathematical solutions to partial differential equations (PDEs) that appropriately simulate complex real-world problems including stress/strain testing, thermal conduction, and mass transport in various geometries and materials. In this study, the FEM allowed us to predict the concentration gradient with respect to time depending on the quantity of PODS®-rhBDNF introduced into the system. To solve the FEM, we used COMSOL® Multiphysics (version 5.6, released on Nov. 11, 2020, COMSOL, Inc., Burlington, Massachusetts, USA), which is a finite element method solution tool for engineering and scientific research computations. We used sustained-release kinetics for PODS®-rhBDNF determined from aforementioned ELISA and SDS-PAGE experiments, as well as data from a previous study by our group. Device geometry was constructed at a 1:1 scale using Autodesk® Inventor 2019.0.2 (Autodesk, Mill Valley, CA, USA) (panel B of FIG. 3). The FEA was computed with a high-performance desktop computer platform equipped with a 64 GB RAM CPU (AMD Ryzen Threadripper 3990X 64-Core, 128-Thread @ 4.3 GHz) and two GPU cards (NVIDIA GeForce RTX 3080Ti, 12 GB 384-bit GFF6X Graphics card).

Human pluripotent stem cell culture using dual-compartment microfluidic device: Human embryonic stem cells (hESCs: H7 and H9, passage number 25-35) were obtained from WiCell Research Institute (Madison, Wisconsin, USA). Human induced pluripotent stem cells (hiPSCs: TC-1133HKK, passage number 22-35) were generated from human CD34+ cord blood cells using the four Yamanaka factors at Lonza (Walkersville, Maryland, USA). The hiPSC cell line (TC-1133HKK) was kindly provided by Healios K.K. (Tokyo, Japan). hPSC-derived ONPs were generated based on our previously established protocol (Supplementary Data). A step-wise series of ligands and growth factors was added to the neuronal induction medium to promote hPSC differentiation toward the late-stage ONP lineage—the mitotic progenitor population that generates SGNs. (FIGS. 3A-3B).

Microfluidic devices provided a platform for specifically evaluating axonal regeneration. The multi-chamber micro uidic culture device we used, Xona™ Micro uidics XC150 and XC450 (Xona™ Micro uidics, Research Triangle Park, North Carolina, USA) (panel A of FIG. 3), mimics the anatomy of the inner ear in regards to volume and general geometry of the microgroove channel array: the bony wall (osseous spiral lamina [OSL]) separating the scala tympani and the modiolus (where extant SGNs are located) contains perforations (Canaliculae Perforantes of Schuknecht [CPS]) with diameters of 5-30 μm (panel B of FIG. 3). Although it is difficult to determine the exact distance between the endogenous SGNs and transplanted SGNs in the scala tympani, we estimate it to be 300-700 μm in distance. The geometry of the XC450 device allows it to reasonably simulate this complex anatomical environment as the length of microgroove channel is 450 μm, approximate the distance between distance between the endogenous SGNs and transplanted SGNs in the scala tympani (see panels A-C of FIG. 3). Moreover, the microgroove channel array allows for neurites to grow toward growth factors in the opposite compartment while limiting migration of derived late-stage ONP cell bodies, as the OSL does to extant SGNs in vivo. The devices were washed and coated with poly-L-ornithine (PLO, 20 μg/mL in H₂O, Sigma-Aldrich, St. Louis, Missouri, USA) and recombinant laminin-511 (iMatrix-511, 0.5 mg/mL, Nacalai USA, San Diego, California, USA) according to the manufacturer-outlined protocol. Next, approximately 1.75×10⁵ cells (in 20 μL of media) were plated in the somal compartment (SC) through the top and bottom left wells (i.e., total amount of 3.5×10⁵ hPSC-derived ONPs were added).

The FEA-determined quantity of PODS®-rhBDNF crystals were deposited in the top right well of the neurotrophin compartment (NTC, panel C of FIG. 3, c-d). hPSC-derived ONPs were cultured for 7 days in the Xona™ device to induce otic neuronal differentiation and neurite growth. Note that high-density cell cultures were induced to facilitate molecular studies as well as the generation of a more biologically relevant neuronal phenotype (i.e., otic lineage). Media was topped off daily after imaging (from 20-40 μL per well). Experiments were done in three biological replicates unless otherwise specified in figure captions.

Immunocytochemistry and image acquisition: On Day 7, 4% (w/v) paraformaldehyde (PFA) (ThermoFisher Scientific, Waltham, MA, USA) was added to the compartments for 20 minutes to fix the cells. Immunocytochemistry was used to assess GATA3, PAX8, and β-III tubulin, and synapsin 1/2 expression. Our previous studies indicate that GATA3 and PAX8 can be used as descriptive markers for the ONP lineage, -III tubulin can be used as a SGN marker, and synapsin 1/2 can be detected at the synapses.

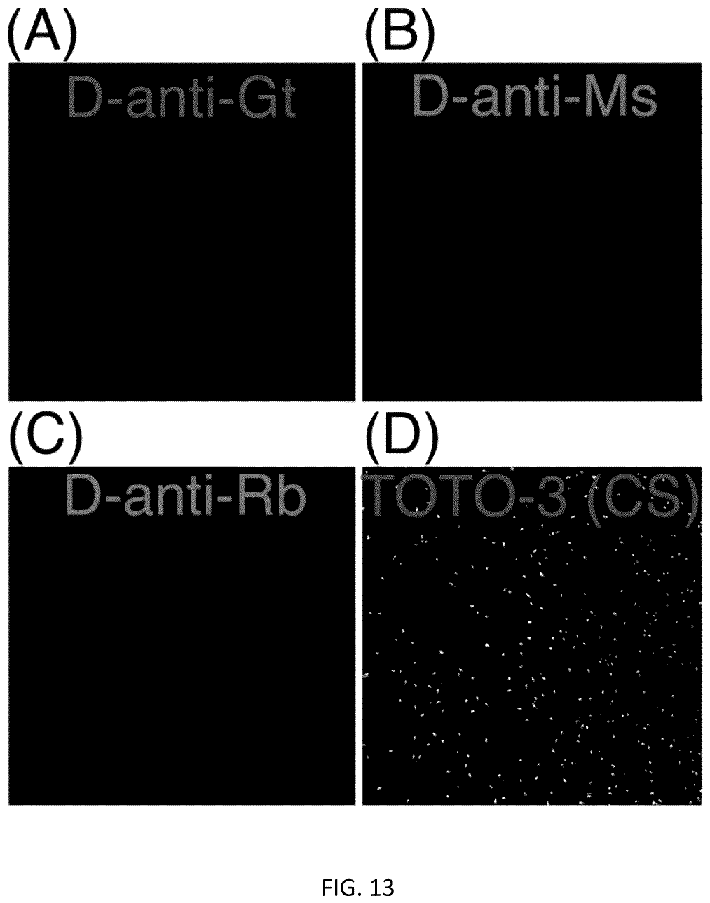

Following PBS washes, cultures were blocked with 5% BSA at room temperature for 1 hour. Cultures were then incubated overnight at 4° C. on a shaker plate in a primary antibody solution using Mouse or Rabbit anti-β-III-tubulin (1:100, Abcam, Cambridge, MA, USA), Goat anti-PAX8 (1:500, Abcam, Cambridge, MA, USA), Mouse anti-GATA3 (1:500, R&D Systems, Minneapolis, MN, USA), or Chicken anti-synapsin 1/2 (1:500, Synaptic Systems, Coventry, United Kingdom). Following PBS washes, cultures were incubated at room temperature for 90 minutes on a shaker plate in secondary antibody solution composed of Alexa uor 647 anti-Rabbit (1:1000, ThermoFisher Scientific, Waltham, MA, USA), Alexa uor 488 anti-Goat (1:1000, ThermoFisher Scientific, Waltham, MA, USA), Alexa uor 594 anti-Mouse (1:1000, ThermoFisher Scientific, Waltham, MA, USA), or Alexa uor 488 anti-Chicken (1:1000, ThermoFisher Scientific, St. Louis, MO, USA) in 1% BSA. Following PBS washes, cultures were incubated with DAPI (300 nM, ThermoFisher Scientific, Waltham, MA, USA) for 20 minutes. Secondary antibody controls were performed each time multiple primary antibodies were used. Labeling controls (detection controls) were performed for a sample from each hPSC culture. See FIG. 13 for exemplary figures for these control conditions. Results were imaged using a Nikon Ti2 Widefield Laser Microscope System (Nikon, Tokyo, Japan). Phase-contrast images were captured on a Nikon Eclipse TE2000-U inverted microscope (Nikon, Tokyo, Japan). All fluorescence images were acquired on a Zeiss LSM 510 META laser-scanning confocal microscope (Carl Zeiss, Oberkochen, Germany), a Nikon Ti2 laser laser-scanning confocal microscope (Nikon, Tokyo, Japan), a Leica SP5 laser-scanning confocal microscope (Leica, Welzlar, Germany), or a Keyence BZ-X810 All-in-One uorescence microscope (Keyence, Osaka, Japan). Observers were blinded to the conditions during imaging and tracing. In general, the images were processed in ImageJ ver. 1.53 g or Matlab R2020b. Further detail on image acquisition and quantification of fluorescent-positive cells can be found in the Supplementary Data.

Lentiviral transduction to generate RFP-postive ONP clones: To generate RFP+hPSC-derived late-stage ONP clones, hPSC-derived early-stage ONPs were transduced with pLenti TurboRFP BlastR (Plasmid #102343, Addgene, Watertown, MA, USA) using the psPAX2 packaging plasmid (#12260, Addgene, Watertown, MA, USA) and pMD2.G VSV-G envelope expressing plasmid (#12259, Addgene, Watertown, MA, USA) and Lipofectamine Stem (STEM00003, Thermo Fisher Scienti c, Waltham, MA, USA). Early-stage ONP clones were selected after 48 hours using puromycin (1 g/mL) to obtain a homogenous culture and were subsequently visualized using uorescence microscopy to con rm expression of the TurboRFP transgene. Early-stage ONP clones were expanded for 72 hours and were subsequently differentiated into late-stage ONPs using our previously established differentiation protocol.

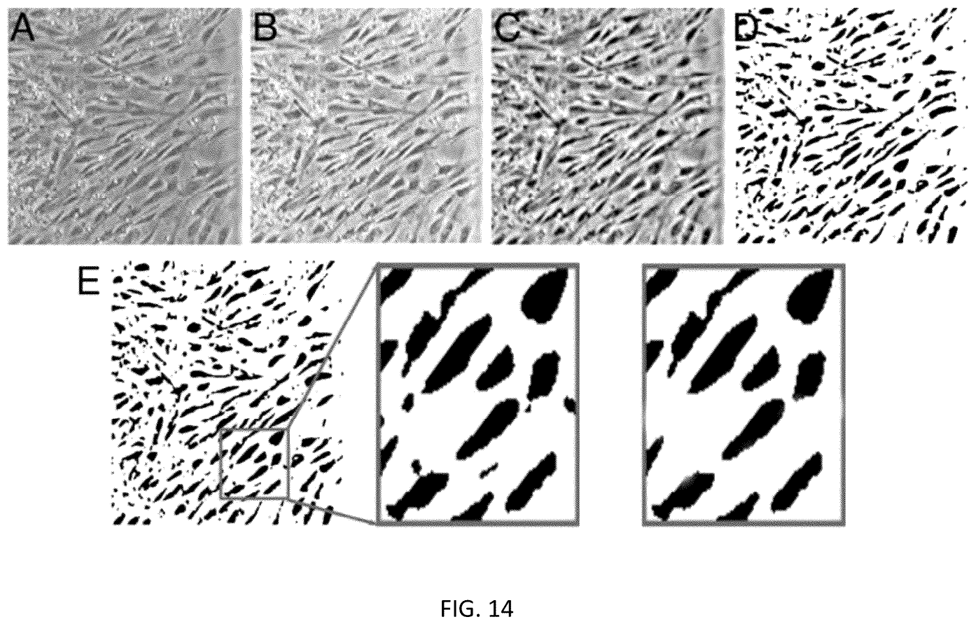

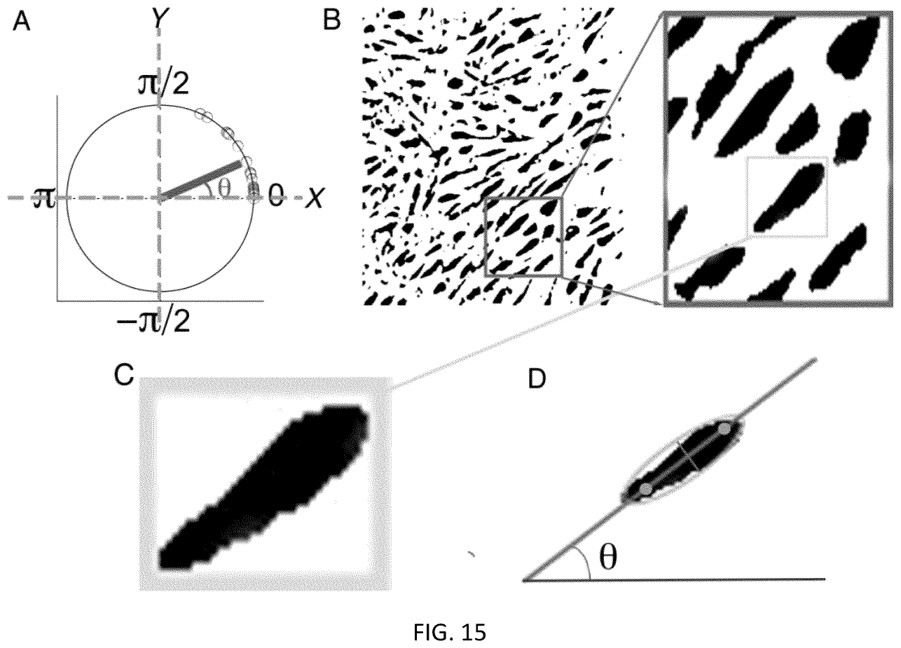

Preferred cell orientation analysis: Collective cell migration, where cells organized in a tightly connected fashion migrate as cohesive structures, is a critical biological process to highlight the neurotrophin diffusion profile. To evaluate this process, time-lapse acquisition of images of the Xona™ device was performed using an inverted microscope (Nikon Eclipse TS100, Tokyo, Japan) on days 1, 3, 5, and 7. Due to the high cell density required for hPSC-derived late stage ONPs to survive in the somal compartment of the Xona™ device, images were not amenable to manual analysis in most of the cases. To circumvent this problem, we performed a series of image pre-processings that are mainly based on modified binarization-based extraction of aliment score methods with some modifications. We used MATLAB Image Processing Toolbox R2020b (version 9.9.0.1495850, Sep. 30, 2020, Mathorks, Natick, MA) for analysis. Please refer to FIG. 14 for further detail. The analysis of directional data in general represents a particular challenge: there is no reason to designate any particular point on the circle as zero, and it is somewhat arbitrary depending on where one sets a coordinate. In this study, we used polar coordinates to determine the directionality of preferred cell orientation. For this analysis, we again used MATLAB Image Processing Toolbox R2020b. See detailed discussion on how we determined the preferred cell orientation in FIG. 15.

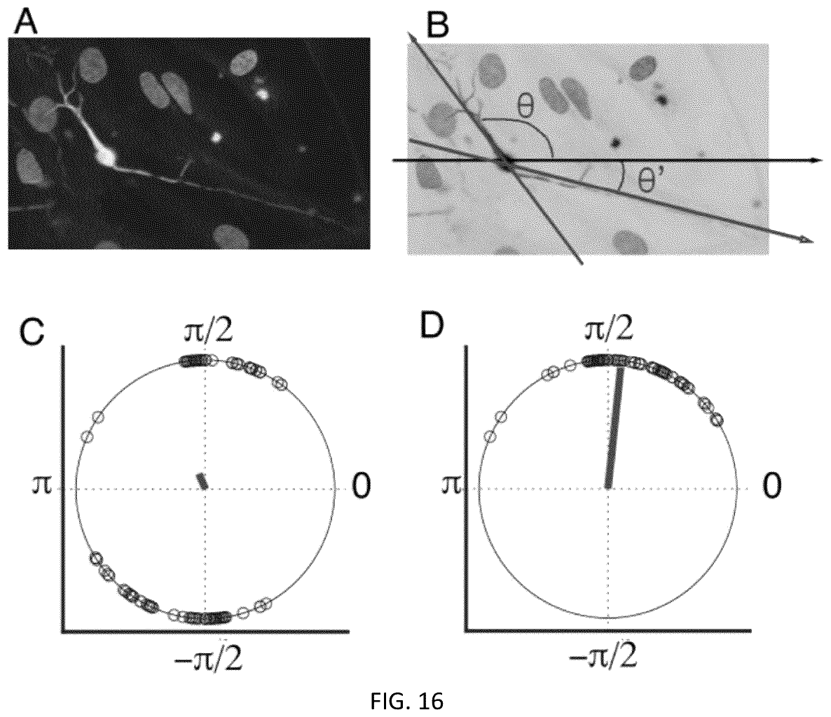

Neurite alignment vector assay, neurite growth assay and cell migration assay: The microfluidic device allowed us to culture hPSC-derived ONPs in a polarized manner and to directly isolate/analyze neurites. To evaluate neurite projection into the neurotrophin compartment by derived otic neurons cultured in the somal compartment, we performed a neurite alignment vector assay. We also evaluated the length of neurites that grew from hPSC-derived ONPs. For these purposes, hPSC-derived ONPs were cultured in a Xona™ XC450 for seven days and were then immunostained with β-III tubulin and DAPI. We used two ImageJ plug-in toolkits, NeuriteTracer and NeuronJ, to assess neurite alignment and neurite growth. Due to the bipolar nature of hPSC-derived ONPs/SGNs, we measured the two longest neurites from each cell. Please refer to FIG. 16 for detailed description of this analysis. We used hPSC-derived ONPs cultured with 800,000 PODS®-rhBDNF crystals as a positive control. This quantity was selected based on our FEM in that concentrations were so high that there was no biologically relevant concentration gradient. As a negative control, we used 20 ng/mL of recombinant human BDNF (standard culture condition). To evaluate cell migration across the microgroove channels, we performed cell migration analysis. We manually counted the number of cells that migrated from the somal compartment into the microgroove channels and neurotrophin compartment.

Statistical analysis: When appropriate, and as indicated in each figure, statistical analysis was performed. Experimental values are typically expressed as mean and standard error (SE). The majority of the statistical analyses were performed with Python 3.9.6. (Python Software Foundation, Wilmington, Delaware, USA). The following libraries were used for the statistical analysis: Scipy, Numpy, Matplotlib, and Seaborn. Normal distributions were assumed unless otherwise mentioned. P values smaller than 0.05 were considered statistically significant. For circular statistics, we derived the sample mean vector and its polar coordinate. Mean and confidence intervals were calculated. We chose confidence coefficient, Q (e.g., Q=0.95). To analyze the axial nature of the data, especially to compute the mean vector angle, we doubled each angle and reduced the multiples modulo 360°. Please see detailed discussion in FIGS. 14 and 16. Rayleigh test of uniformity and V-test were performed to determine whether the samples differ significantly from randomness (i.e., where there is statistical evidence of directionality). One-sample test for the mean angle was performed to test whether the population mean angle is statistically different from the given angle. In all of our circular statistics, von Mises distribution was assumed and also verified. Circular statistics were performed using CircStat: A MATLAB Toolbox. Please see detailed description of circular statistics in FIGS. 14 and 16.

Differentiation of human embryonic stem cells into late-stage otic neuronal progenitors: Undifferentiated human embryonic stem cells (hESCs: H7 and 119) (passages number 25-35) were obtained from WiCell Research Institute (Madison, WI, USA). Human induced pluripotent stem cells (hiPSCs: Cell line: TC-1133HKK) (passage number 22-35) were generated from human CD34+ cord blood cells using the four Yamanaka factors at Lonza (Walkersville, Maryland, USA). The hiPSC cell line (TC-1133HKK) was kindly provided by Healios K.K. (Tokyo, Japan). Karyotypes of hESCs and hiPSCs were tested at WiCell Research Institute and they were also tested for mycoplasma using MycoAler™ Mycoplasma Detection Kit (Lonza, Basel, Switzerland). Human pluripotent stem cells (hPSCs: ESCs and hiPSCs) were cultured in StemFit™ BasicO2/O3 (Ajinomoto Co., Inc. Tokyo, Japan) or mTeSi™ 1(#85850; STEMCELL Technologies, British Columbia, Canada) on iMatrix-5117 (#892012; Nacalai USA, California, USA). Cells were purified using Anti-TRA-1-60 microbeads, a Neural Crest Stem Cell MicroBeads Kit, LS Columns, and a MidiMACS Separator (Miltenyi Biotec, Bergisch Glad-bach, Germany). After seeding purified cells into a chemically defined medium containing 1% (v/v) N2 supplement-A (#07152; STEMCELL Technologies, BC, Canada), 2% (v/v) NeuroCult™ SM1 supplements (#05711, STEMCELL Technologies, BC, Canada), and 100 μM beta-mercaptoethanol (#21985923; Thermo Fisher Scientific, Waltham, MA, USA), a stepwise series of ligands and growth factors were added to promote hESC differentiation towards a late-stage ONP lineage (See FIG. 3).

Human ESC derived late-stage ONPs were then expanded in the ONP maintenance medium (1:1 Neurobasal medium [#21103049, Thermo Fisher Scientific, Waltham, MA, USA]/DMEM-F12 medium [#51445C, MilliporeSigma, St. Louis, MO, USA]+20% StemFit™ for Differentiation EAS4011 (Ajinomoto Co., inc. Tokyo, Japan)) supplemented with 10% fetal bovine serum (FBS) (#10437028, ThermoFisher Scientific, Waltham, MA, USA) and received three human growth factors—human recombinant EGF (#78006, STEMCELL Technologies, BC, Canada), human recombinant FGF2 (#78134, STEMCELL Technologies, BC, Canada), and human recombinant IGF-1 (#78142, STEMCELL Technologies, BC, Canada) until they reached passage-number three to five. hESC-derived late-stage ONPs were then incubated with Accutase™ (STEMCELL Technologies, BC, Canada) for 3.5 minutes to facilitate detachment. After centrifugation and cell counting, cells were re-suspended into the neuronal induction medium, which is composed of Brainphys™ Neuronal Medium (#05790, STEMCELL Technologies, BC, Canada), 1% (v/v) N2 supplement-A, 2% (v/v) NeuroCult7 SM1 supplements, 1 mM Glutamax™ Supplement (#35050061, Thermo Fisher Scientific, Waltham, MA, USA), supplemented with 10% FBS and 2 mg/mL of bovine serum albumin (BSA) (#23209, Thermo Fisher Scientific, Waltham, MA, USA).

Image acquisition and quantification of florescent-positive cells: The level of fluorescence in a given region (e.g., nucleus) was determined using the corrected total cell fluorescence (CTCF) based on the following formula. CTCF=Integrated Density−(Area of selected cell×Mean fluorescence of background readings). DAPI-stained cells were counted using the ITCN (image-based tool for counting nuclei) plugin for ImageJ developed by Thomas Kuo and Jiyun Byun at the Center for Bio-image Informatics at the University of California Santa Barbara. Images were converted to 8-bit grey scale and inverted before using ITCN. Cell detection was performed detecting dark peaks with the following parameters: cell width=7, minimum distance=7, threshold=2, mask image: use selected ROI. The number of positively labeled cells was counted in 10 random selected fields and was expressed as a percentage of the total number of cells counted. The total number of cells was quantified by DAPI nuclear staining.

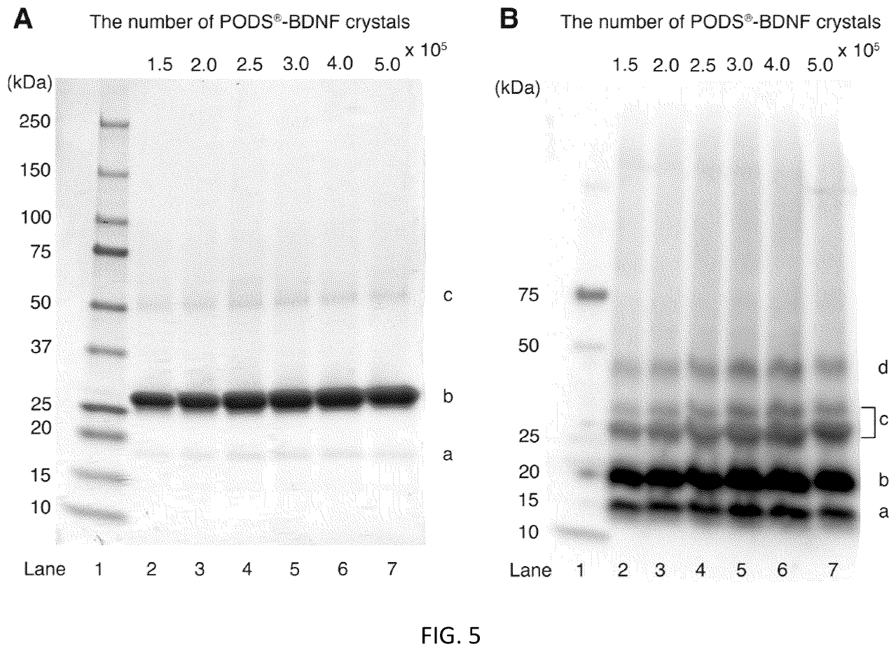

Dimension reduction in diffusion flux: justification: To reduce the computational intensity, we reduced a dimension from 3D to 2D to compute diffusion Flux. The goal of dimension reduction here is to find a representation of a Cartesian Coordinate system that allows to project the 3D data vectors on it and obtain a 2D representation of data. The dimension reduction was justified by 1) mono-layer nature of hESC-derived ONP culture in a Xona™ device. 2) A thickness of microgroove channel was 3 μm and that of a soma channel was 10 μm, both of the values were fewer than the size of an ONP. Using COMSOL Chemical Engineering Module, diffusion fluxes were computed at Day 1, 3, 5, and 7. Also note that, as shown in FIG. 5, the diffusion flux was stable from Day 1-7.

Image processing strategies: Due to the high cell density required for hESC-ONPs to survive in a Soma chamber, images were not amenable to manually analyze as it was difficult to distinguish individual cell bodies. To circumvent this problem, we performed the following steps mainly based on binarization-based extraction of aliment score (BEAS) with some modifications.

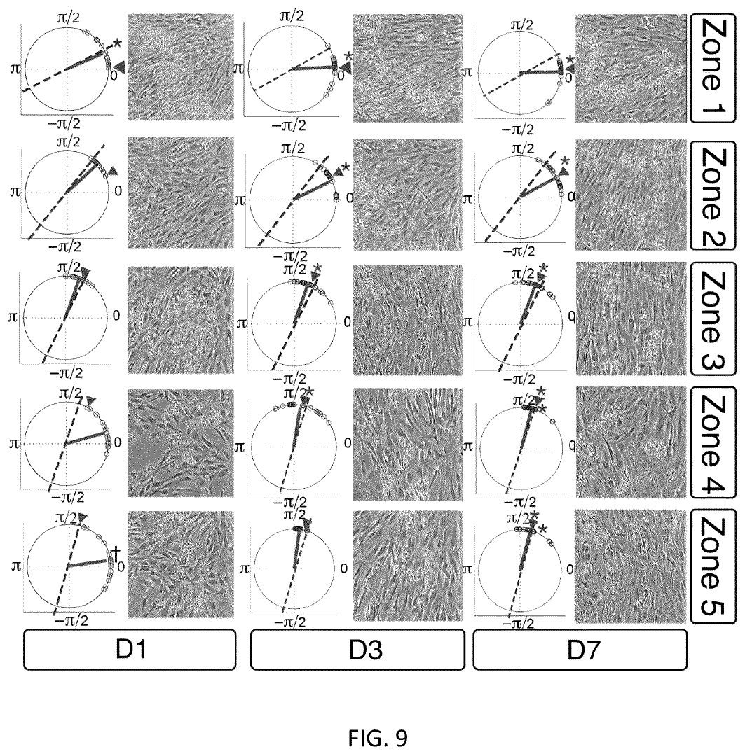

Step 1: Obtaining images: We obtained phase-contrast microscopic images of hESC-derived ONPs cultured in Xona™ XC450 (Xona™ Microfluidics, Research Triangle Park, North Carolina, USA) for computational calculation and biological validation (FIGS. 13B-13C) using Nikon ECLIPSE TS100 (Nikon, Tokyo, Japan). The images were obtained at DIV 1, 3, and 7 to generate a time-series sequence (panel A of FIG. 14 and also FIG. 9).

Step 2: Despeckle Filtering: The following 4 steps required MATLAB R2020b version 9.9.0.1495850 (The MathWorks, Inc., Natick, MA, USA). The image was first processed with De-speckle filtering using Image Despeckle Filtering Toolbox. The Despeckle filter detect the edges of individual cells in a layer and blurs all of the selection except those edges. This blurring removes noise while preserving detail on each image (panel B of FIG. 14).

Step 3: Band-pass Filtering: Band-pass-filter attenuates signal frequencies outside a range of interest. To decrease noise on images at the same time reducing low-frequency artifact, band-pass filters were used to further delineate image features such as cell edges. Gaussian Band-pass Filter for Image Processing for MATLAB R2020b was used for this process (panel C of FIG. 14).

Step 4: Sauvola local image thresholding: Sauvola binarization method is well suited for image binarization. This process was performed by using Sauvola local image thresholding for MATLAB R2020b (FIGS. 13D-13E. Red highlight area on panel E of FIG. 14 was magnified and shown in the middle of the second row.

Step 5: Filling image regions and holes: A MATLAB function “imfill” was used to fill image regions and holes. This was based on an algorithm on morphological reconstruction so that cell morphology can be detected more easily with less noise (i.e., small binary bits). An explanatory image was shown on the right side of the second row in FIG. 14).

Preferred cell orientation: Cell preferential orientation is one of the salient features to highlight neurotrophin diffusion gradient profile. Therefore, we first studied the preferential orientation of the hESC-derived ONPs by plotting the distribution of their directional angle data. The analysis of directional data in general represents a particular challenge: There is no reason to designate any particular point on the circle as zero, just as it is somewhat arbitrary depending on where one sets a coordinate. We used the polar coordinate to determine the directionality of cell orientation (panel A of FIG. 15). In the polar coordinate, we used the polar angle denoted by θ in this study (panel A of FIG. 15). Here, the polar angle (θ) is defined as an angle from the positive X axis in a rectangular coordinate system in the counter-clock wise direction (panel A of FIG. 15). Note that in some cases, a polar angle may exceed the interval from 0° to 360° and hence, the angle has to be reduced modulo 360°.

The orientation of any given cell was defined as the orientation of the major axis of the best fit ellipse. the orientation of the cells was measured by angle ranging from 0°-360° or from 0-2π radians. Note that we studied the orientation cells by plotting the distribution of their angle in relation to 0° (shown in blue horizontal line in panel D of FIG. 15). Preferred orientation of any individual cell, expressed in a polar angle θ, was defined as the orientation of the major axis of the best-fit ellipse, which is typical morphology of hESC-derived ONPs (panel B of FIG. 14, magnified in panel C of FIG. 15).

To automate this process, A MATLAB Image Processing Toolbox function, “regionprops” was used to measure the angle (in degrees and radians) between the x-axis and the major axis of the putative ellipse that has the same second moments (See panel D of FIG. 15 for a cell example we used for this analysis). To compute this value automatically, we used a MATLAB property called “Orientation”. The direction and length of the ellipse's axes are given by the eigenvectors and eigenvalues of the covariance matrix. Panel D of FIG. 15 illustrates the axes (the longer red line) and orientation of the ellipse that represents cell orientation. The red lines show the axes (major and minor axis) and the gray dots are showing the foci. The cell orientation angle was defined as the angle between the horizontal line (dark blue line) and the major axis (the longer red line) denoted by 0.

Neurite growth analysis: Automated neurite growth analysis was performed using Image J. Neurite outgrowth assays are among the most commonly utilized methods to evaluate axonal regeneration. To analyze derived hPSC-derived ONPs neurite outgrowth, the cells were fixed and immunostained for a cytoskeletal marker, the neuron-specific β-III tubulin protein to fill the cell body and neuronal extensions, allowing to automatic imaging and analysis of neuronal morphology including parameters such as neurite count, and neurite length.

Step 1: Automatic quantification algorithms for measuring neurite outgrowth: It has been demonstrated that human type-I SGNs and hESC-derived SGNs were both essentially bipolar neurons (panels A-B of FIG. 15. In this study, we, therefore, analyzed the two longest neurites arising from a cell body. In majority of the images, we were able to use an Image-J plug-in toolkit, NeuriteTracer and also the ImageJ plug-in NeuronJ for tracing neurite alignment and also for automated neurite quantification. Pair of images corresponding to nuclei (DAPI, TOTO-3, or PAX8) and neuritetubulin) were required. It was sometimes necessary to pre-process an image with noise reduction with low-pass filter deconvolution, and shading correction. Soma segmentation was occasionally required for intensity thresholding and morphological filtering. These were also performed with Image J.

Step 2: Neurite alignment vector analysis: Given the bipolar nature of hESC-derived ONPs and SGNs, we assumed that the distribution of our samples are bimodal. As a result, the mean vector, r, would contain little information (panel C of FIG. 16, red line representing the mean vector r). To analyze such data in circular statistics, we had to relate the period of 180 degrees with a full turn much in the same way as we relate to 24 hours with 360 degrees.

To adequately handle data that have bimodal distribution such as our data set, the method of doubling the angles has been widely used in geology and more recently in biology. We, therefore, analyzed our data with this method. To apply this technique to our data, each angle was doubled so as to reduce the multiple modulo 360 degrees. This manipulation resulted in unimodal circular distribution. The biomodal directions are now clustered around unidirectional distribution: resulting in more meaningful mean vector r (shown in panel D of FIG. 16).

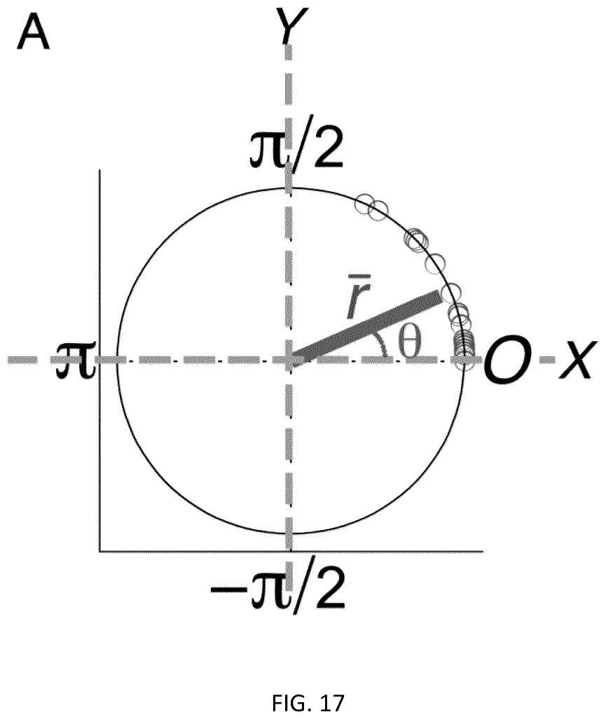

Descriptive statistics: Measure of location: Mean vector angle: One way to estimate preferred orientation of the cell is to calculate the mean vector angle. We defined a mean vector angle as follows. Given the nature of circular statistics, the arithmetic mean of angles fail to be applicable. In this instance, we used the polar coordinate system with X and Y axis and origin O (panel A of FIG. 17). Let θₙ, be one of the n observed angles obtained from cell orientation analysis (blue circle). Here, we are only interested in angle; not the vector length, hence, we set the corresponding unit vector as 1 until otherwise noted. By definition of elementary trigonometric function, the vector rᵢ can be defined as follows.

xᵢ=cos(θᵢ),yᵢ=sin(θᵢ)

Panel A of FIG. 17 illustrates that all of rᵢ lie on the unit circle where an unit vector of 1. Also note that the x-coordinate of a point corresponds to the cosine of the angle and the y-coordinate to the sine of the angle. We then averaged rᵢ as shown below.

After this transformation, the vectors r, are vector averaged by

$$\overline{r} = \frac{1}{N} {\sum }_{i = 1}^{n} \overline{r}$$

Here, the vector r is defined as mean directorial vector. Preferred cell orientation angle θ can be calculated using an inverse tangent function.

Measures of Dispersion/Concentration: Since our samples were unimodal distribution for preferred cell orientation data, the mean vector length, r, serves as measure of concentration. Angular variance and standard deviation were also computed with CircStat: MATLAB Toolbox.

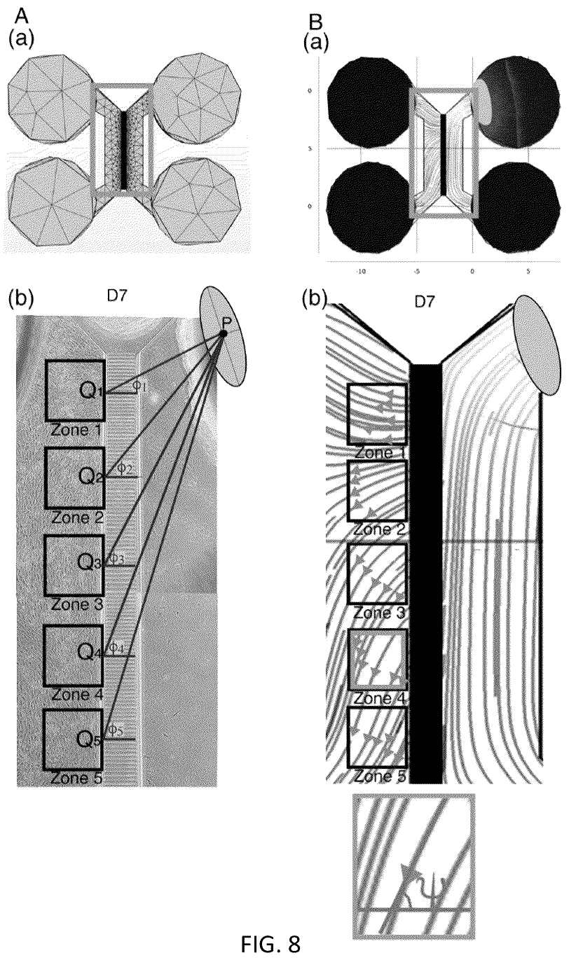

The one-sample test for the mean angle: Briefly, we assume here that the samples are reasonably unimodal and symmetric (FIG. 8) with respect to the median. We then consider that the samples are distributed with a von Mises distribution. We wish to determine the confidence limits for the mean vector angle θ. For this purpose, we first determined the mean vector length r (FIG. 23) and the mean vector angle θ. Once we determine r and θ, we chose confidence coefficient Q=0.05. We then finally identify an angle of deviation, 6. The desired confident upper limit is θ+δ and the lower limit is θ−δ. Using θ, we tested our hypothesis—whether the sample mean angle θ differs significantly from EDAs (φ) or DFAs (ψ). Let us set Ho: The mean vector angle has a mean of φ. If we determine confidence limit θ+δ, and also if φ lies inside the confidence interval, we can conclude that we may not reject H₀. Thus, we can conclude that the mean vector angle does not differ significantly from EDAs or DFAs.

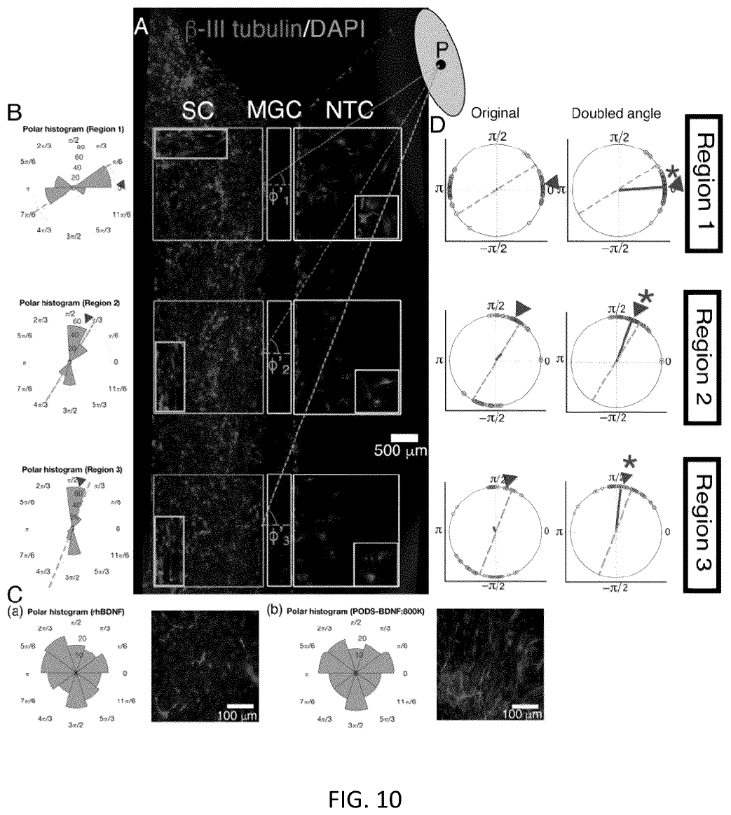

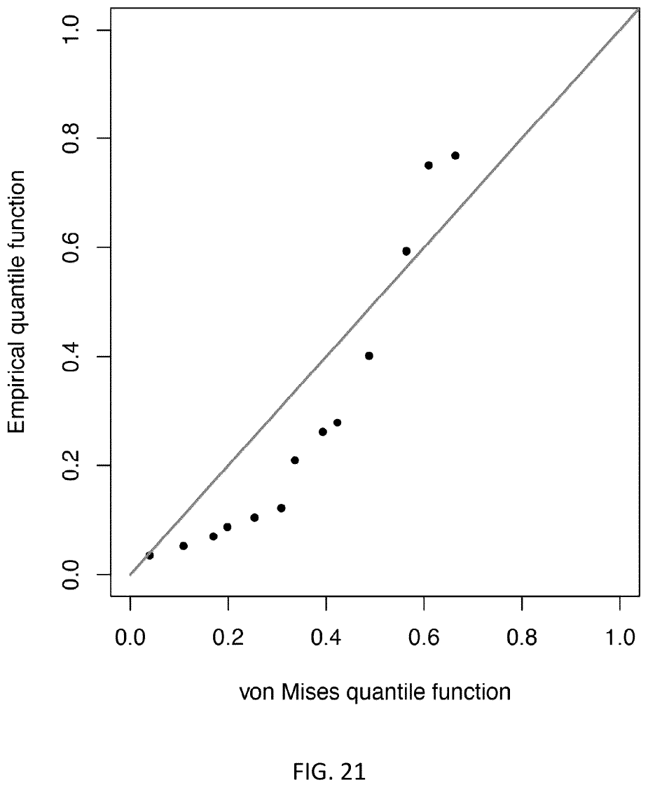

von Mises distribution: To perform the Rayleigh and the V tests, data are required to distribute with a von Mises distribution—a Gaussian distribution equivalent on Circular statistics. We therefore verified all of our data with a graphical assessment of goodness-of-fit for von Mises distribution using the Quantile-Quantile plot (Q-Q plot) (FIG. 21). We used an R library, Clair/AS.circular package: ArchStats (circular function) based on ‘Circular Statistics in R’. This method requires preliminary estimate fork and p of the parameter K and μ of the von Mises probability distribution function. k and μ were computed via maximum likelihood method using a MATLAB function called “circ_vmpar” in CircStat: A MATLAB Toolbox for Circular Statistics. FIG. 21 demonstrates von Mises Q-Q plot for the data pertinent to preferred cell orientation angle in Zone 1 on Day 1. As seen in this figure, the plot was scattered around a line passing through (0, 0) with a slope of 45°. All of the data used for FIGS. 9 and 10 were analyzed with this method and they were graphically confirmed the von Mises distribution. Note here that we eliminated “discordant observation”, which is judged to be significantly far away from the main data mass on the basis of graphical analysis of the Q-Q plot. Due to the nature of our automated analyses, these errors are unavoidable. We rarely omitted the outliers and outliers in circular data in general do not usually present serious problems to the data analysis.

### Results

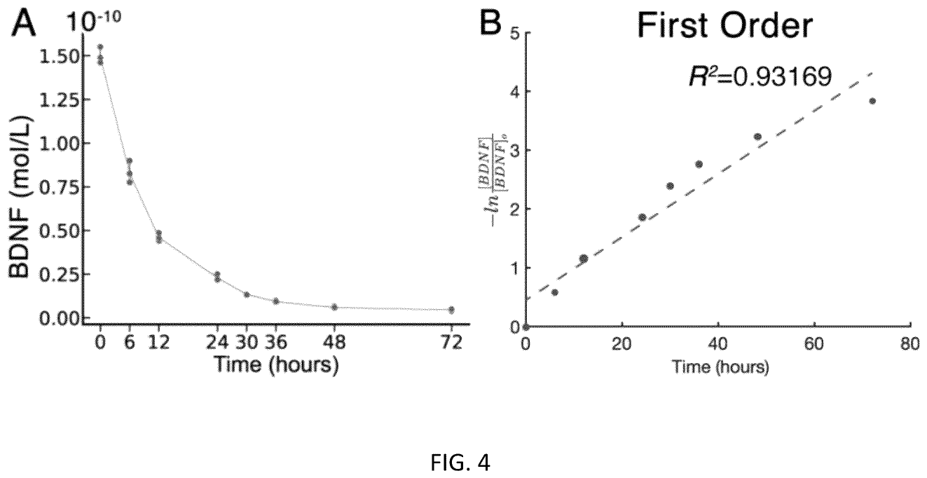

The appropriate quantity of PODS®-rhBDNF crystals to induce an effective neurotrophin gradient for otic neuronal differentiation and directed neurite outgrowth was derived using a three-dimensional FEA that predicts the concentration profile of BDNF formed through the gradual release and diffusion of BDNF from PODS®-rhBDNF. First, we quantified the molecular kinetics of this phenomenon with ELISA testing (FIG. 4) to establish parameters for the FEA. Here, two consecutive molecular reactions occur: (1) the breakdown of PODS® crystals into polyhedrin protein and rhBDNF, and (2) the degradation of rhBDNF towards the degradation product (Equation (1)).

$$PODS {\to }^{k 1} BDNF + PHP {\to }^{k 2} DP + PHP \qquad ( 1 )$$

where DP is the degradation product of the released rhBDNF, PHP is the polyhedrin protein, and k₁ and k₂ are the rate constants (1/hour) for their respective reactions.

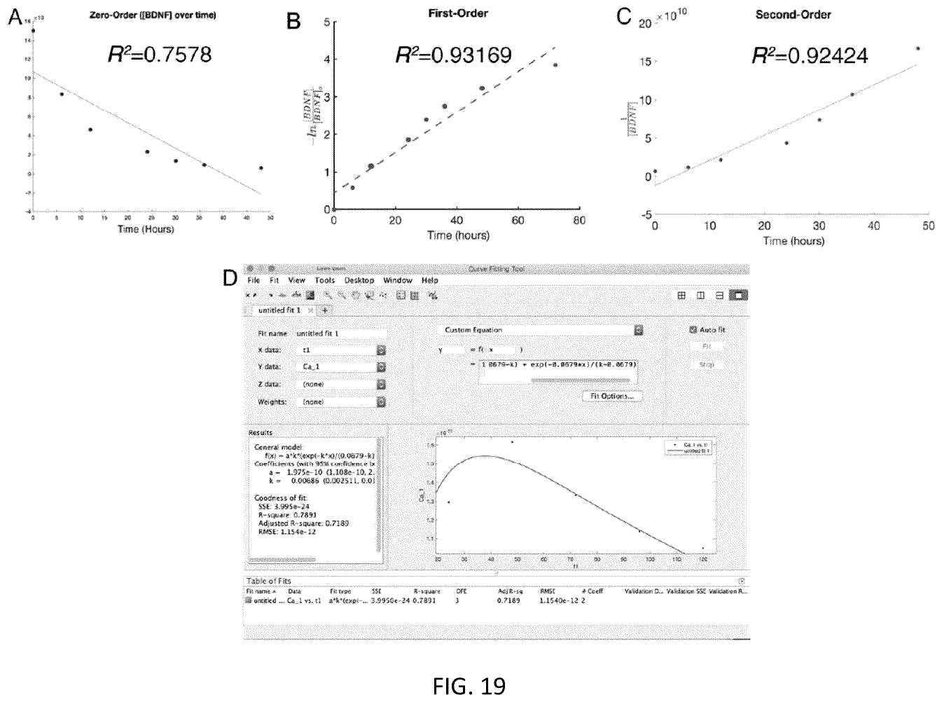

Degradation kinetics data for rhBDNF were collected while monitoring rhBDNF concentration after introducing a predefined amount into a single well of solution. The data obtained throughout the first 72 hours indicate an exponential decay, suggesting first-order kinetics (FIG. 4). To confirm this interpretation, we performed linear and nonlinear least square analyses of the kinetics data with the MATLAB Curve Fitting Toolbox. We found that the corresponding R² for the first-order curve-fit was 0.93169, confirming that rhBDNF degradation in the experimental condition is indeed first-order. The rate constant for a first-order reaction is defined to be the slope of the semi-logarithmic plot of measured concentration (proportional to initial concentration) vs time. The value for k₂ is calculated to be 0.0679 (1/hour) (See further detail in FIG. 19). Furthermore, data for the complete molecular reaction were collected by similarly monitoring rhBDNF concentration over time after placing a predefined quantity of PODS®-rhBDNF crystals into a single well of solution. The data appeared to fit the nonlinear curve of Equation (2), which describes the concentration of the intermediate product of two consecutive first-order reactions:

$${C}_{rhBDNF} = {C}_{PODS} \cdot {k}_{1} \cdot ( \frac{{e}^{- {k}_{1} \cdot t}}{{k}_{2} - {k}_{1}} + \frac{{e}^{- {k}_{2} \cdot t}}{{k}_{1} - {k}_{2}} ) \qquad ( 2 )$$

where C_{rhBDNF} is the concentration of the intermediate product rhBDNF and C_{POPS} is the initial concentration of PODS®.

We successfully fit the data empirically to this nonlinear curve (R²=0.7891), approximating k₁ to be 0.00686 (1/hour) (See further detail in FIG. 19).

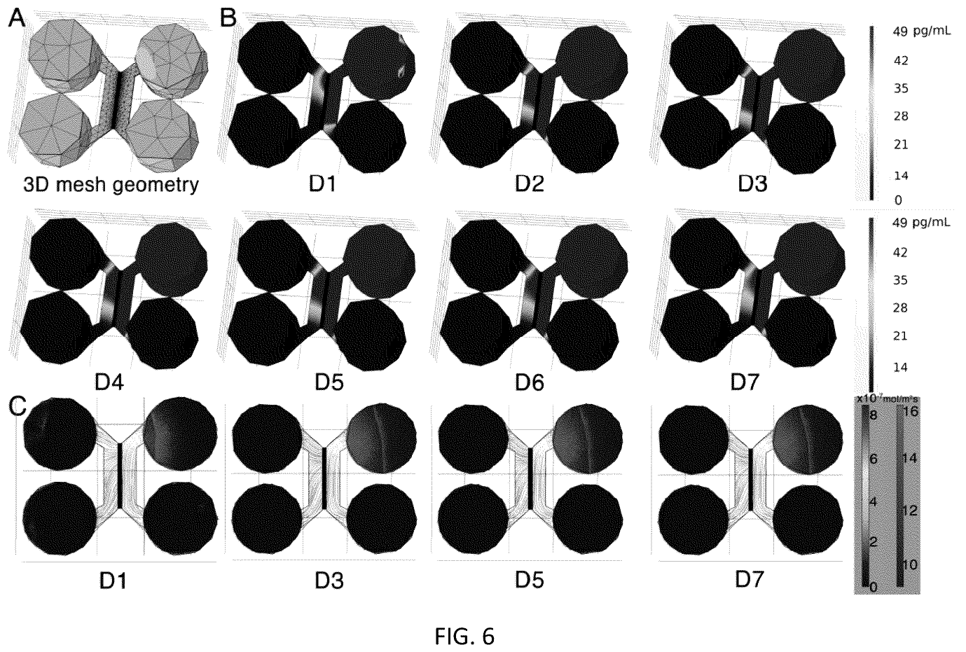

SDS-PAGE was used to separate PODS®-rhBDNF crystals into its constituent proteins to determine the molar ratio of polyhedrin to rhBDNF. Visualization with Coomassie G-250 solution (panel A of FIG. 6) revealed three distinct protein bands at 18.8 (a), 28.0 (b), and 46.8 (c) kDa, which correspond to the molecular weights of Hl-tagged BDNF monomer, polyhedrin, and Hl-tagged BDNF monomer conjoined with polyhedrin, respectively. Western blot analysis was subsequently conducted to confirm the identity of the 18.8 kDa band as rhBDNF. Enhanced chemiluminescence (panel B of FIG. 6) revealed four protein bands at 14.0 (a), 18.8 (b), 37.6 (c), and 46.8 (d) kDa, which correspond with the molecular weights of rhBDNF monomer, Hl-tagged BDNF monomer, Hl-tagged BDNF dimer, and Hl-tagged BDNF monomer conjoined with polyhedrin. Immunoblot detection of the 18.8 kDa band further validates its identity as rhBDNF. SDS-PAGE images were converted to 8-bit grayscale and mean corrected integrated pixel intensity values were calculated for protein bands located at 28.0 and 18.8 kDa; the protein band detected at 46.8 kDa was omitted from the final analysis. Results indicated that the molecular ratio of polyhedrin to rhBDNF is approximately 22:1 in PODS®-rhBDNF. This transforms Equation (1) into:

$$PODS {\to }^{k 1} BDNF + 22 PHP {\to }^{k 2} DP + 22 PHP \qquad ( 3 )$$

With the calculated molar ratio and rate constants for the complete molecular reaction, the resulting chemical gradient with respect to time after PODS®-rhBDNF placement can be solved for any geometry by applying Fick's second Law of diffusion (Equation (4)) and the appropriate boundary (Equations (5) and (6)) and initial conditions (Equation (7)):

$$\frac{dC}{dt} = ∇ \cdot ( D \cdot ∇ C ) - {k}_{2} \cdot C \qquad ( 4 )$$

Boundary Conditions:

δC|_{walls}=0 (5)

C_{BDNF}_{ₚ}_{,i}=Cᵢ (6)

Initial Conditions:

C|ₜ₌₀=0 (7)

where C is the concentration of rhBDNF, D is diffusivity of rhBDNF (6.76 mm²/day), and −k₂·C is the sink term corresponding to the degradation and cell-utilization of the rhBDNF. The first boundary condition (Equation (5)) declares that the concentration of rhBDNF at the walls of the microfludic device are fixed at 0. The second boundary condition (Equation (6)) is a “continuous concentration” boundary condition denoting that the concentration of rhBDNF just behind the boundary of the PODS®-rhBDNF volume (C_{BDNFP,i}) is equal to the concentration of rhBDNF just beyond the same boundary in the surrounding media (Cᵢ).

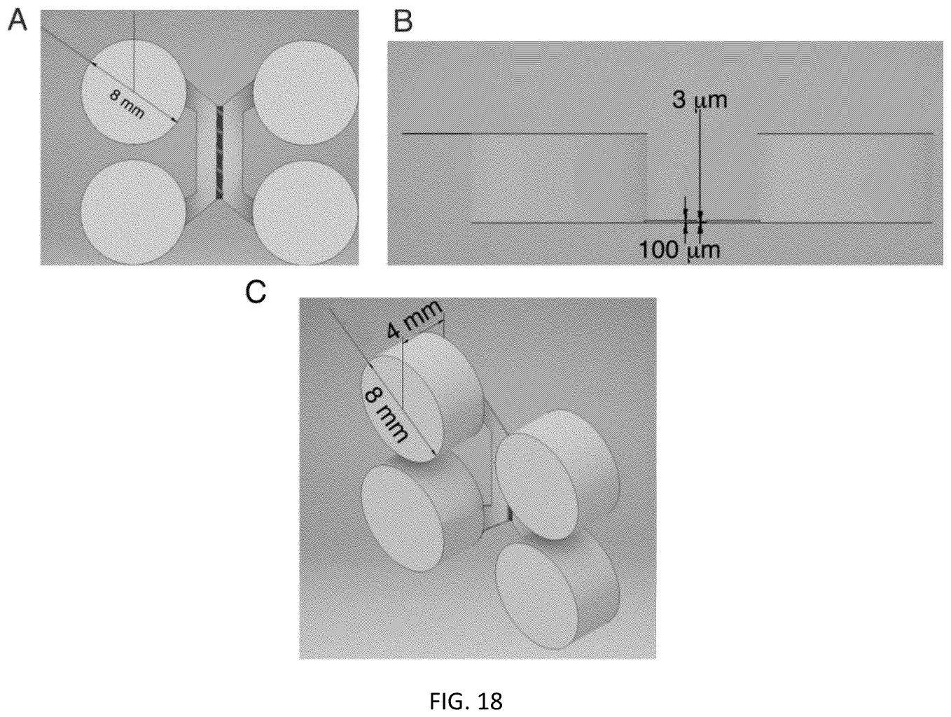

We empirically tested the FEA with two of the available Xona™ devices with differing microchannel lengths, i.e., Xona™-XC150 (the length of the microgroove channels: 150 μm) and Xona™-XC450 (the length of the microgroove channels: 450 μm). This was done first because mass (i.e., BDNF) transport from the neurotrophin compartment (NTC) through the micro-groove channels into the somal compartment (SC) is an important factor in generating the concentration gradient via diffusion mixing. We determined that the Xona™ Microfluidics XC450 was more appropriate for this study as anatomical measurements using an emission electron microscope have demonstrated that the distance from the medial wall of the scala tympani to the SGN cell body is approximately 400 μm. Furthermore, the micro-groove channels in the Xona™ device simulate the pores of the osseous spiral lamina and modiolus (i.e., CPS), which constitutes the separation between the scala tympani and extant SGNs. Please see FIG. 18 for detailed measurements of the three-dimensional mesh of the XC450 generated for the FEA.

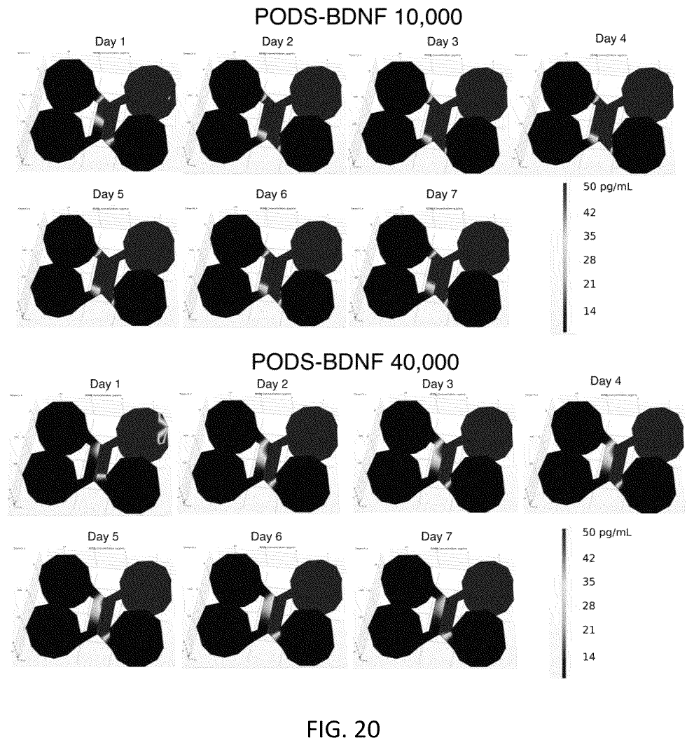

The finite element analysis was then empirically optimized to generate an rhBDNF concentration gradient adequate for hPSC-derived late-stage ONP differentiation toward SGNs and their directed neurite extension by using various PODS®-rhBDNF quantities and time intervals. Panels A-C of FIG. 6 show FEA-computed rhBDNF concentration profiles for 20,000 PODS®-rhBDNF crystals from D1-D7. Note that the rhBDNF concentrations were tuned to be greater throughout D2-D5 to promote the neuronal differentiation and neurite outgrowth observed on D1-D7 (panel B of FIG. 6). Computed diffusion flux was uniform throughout D1-D7 (panel C of FIG. 6). Also note that highest concentration of rhBDNF released from PODS® crystals was greater than 50 pg/mL, the concentration sufficient for otic neuronal differentiation and neurite outgrowth of hPSC-derived ONP 3D spheroids determined with our previously published data. Please see detailed discussion of the empirical optimization in FIG. 20.

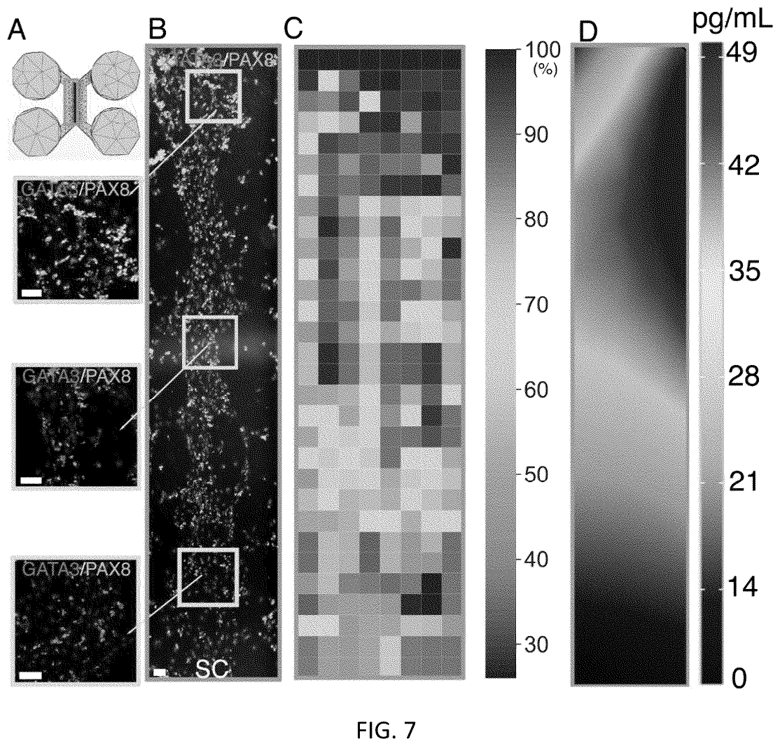

After the biological experiments, we performed quantitative analysis of PAX8 and GATA3 double-positive cells using immunocytochemistry to objectively compare the degree of otic neuronal differentiation in the hPSC-derived ONPs. PAX8 and GATA3 were selected for this analysis in accordance with our previous studies indicating high expression of these protein markers in hPSC-derived late-stage ONPs and early-stage hPSC-derived SGNs. Cells were stained in the somal compartment of the Xona™ device, highlighted in green in panel A of FIG. 7. Panel B of FIG. 7 shows the resulting image of cells in the somal compartment, while a heat-map representation of the percentage of double-positive cells is shown in panel C of FIG. 7. It should be noted that the heat-map is sensitive to the differences in cell density across the channels. This was accounted for by averaging the double-positivity across three biological replicates. The heat-map indicates higher double-positivity in the upper region of the somal compartment (SC), which is closest to the PODS®-rhBDNF disc placement (shown as an orange ellipse in FIG. 7A) in the neurotrophin compartment (NTC). Double-positivity decreases as distance from the PODS®-hBDNF disc increases, supporting the presence and effects of a rhBDNF neurotrophin gradient as predicted by our computational model (panel D of FIG. 7).

We defined two hypothetical directional angles to predict the orientation of hPSC-derived ONPs and neurite growth (FIG. 8). The n-dimensional Euclidean space, denoted by Rⁿ, is a linear vector space in that we can use polar coordinates to compute the directionality of cells and neurites. Here, we used n=1 and 2. For one-dimensional Euclidean space (n=1), we simply drew a line for the Euclidean distance the shortest distance between two points as shown in panel A of FIG. 8, (b) (dark green lines). The two points were (1) the center point of the PODS®-rhBDNF disc (P) and (2) the midpoint of the medial side (Q₁₋₅) (i.e., the near side to microgroove channels) of a pre-designated square (shown as a black square, zones 1-5 in FIG. 8), respectively. The Euclidean distance angle (EDA), φᵢ, was defined as the angle between the horizontal line zero direction and the line PQᵢ that consists of the Euclidean distance where i=1-5.

For two-dimensional Euclidean space (n=2), we utilized Fick's first law, which dictates that the diffusion flux (D) is proportional to the concentration gradient (C):

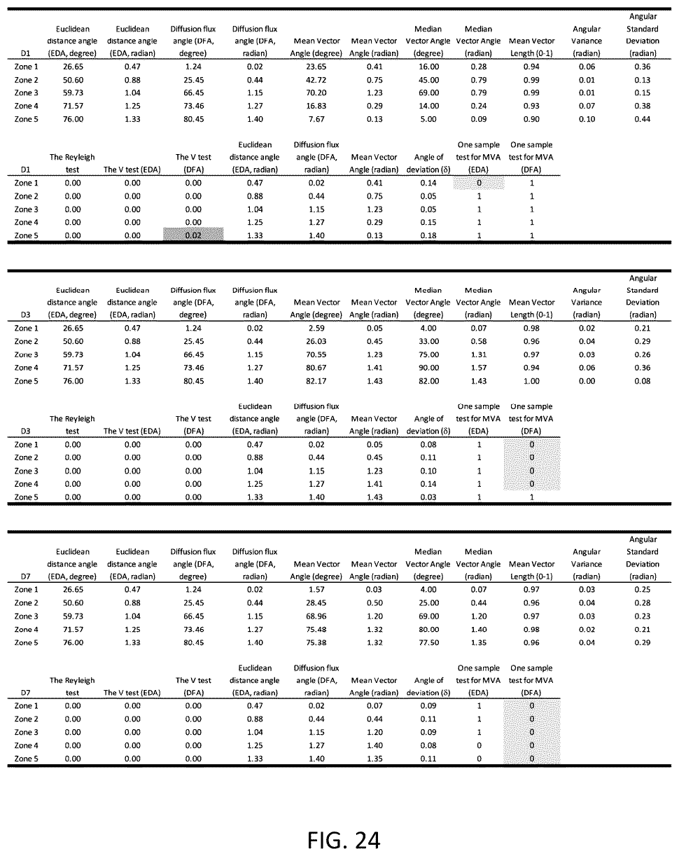

J=D∇C (8)

where J represents the flux vector. Based on this theorem, the direction of a flow vector can be used to represent the directionality of the concentration gradient. We hypothesized that cell orientation is directionally controlled by the flux vector (driven by the concentration gradient). Panel B of FIG. 8 shows the flow vectors in the somal compartment at Day 7 computed by the COMSOL Chemical Reaction Engineering module. We averaged the 10 flow vectors in each of five zones in FIG. 8 to compute the diffusion flux angle (DFA), ψᵢ, where i=1-5 in FIG. 8. To lighten the computational intensity, we reduced the dimension from 3D to 2D to compute diffusion flux. All of the computed EDAs and DFAs can be found in FIG. 24.

FIG. 9 shows time-series of microscopic phase-contrast photomicrographs obtained on days 1, 3, and 7 in the five zones in the Xona™ XC450. Each preferred orientation of any given cell was computed and then plotted on a polar diagram (blue circle). Mean vector angle (MVA, shown as a red line on FIG. 9) and median vector angle were computed. All of the polar diagrams in FIG. 9 show that preferred orientation of hPSC-derived ONPs distribute in an unimodal distribution. We also confirmed that a von Mises distribution is appropriate for these sets of data (See FIG. 21). We performed further analysis to assess if the cells had a tendency to be oriented in a certain direction. We used the Rayleigh test of uniformity to evaluate whether there was statistical evidence of circular directionality. Computed p values for all the 15 conditions were less than 0.05, demonstrating that all of the conditions had statistically significant directionality. Furthermore, to discern whether the observed angles had a tendency to cluster around the two hypothetical angles (i.e., EDA and DFA), we then performed the V test. Likewise, p values for all 15 samples were less than 0.05 except one (Zone 5 on Day 1), underscoring that most of the conditions had statistically significant tendencies to cluster around the Euclidean distance angles (EDAs) and diffusion flux angles (DFAs). Additionally, we performed one sample test for the mean vector angle, similar to a one sample t-test on a linear scale. There was only one condition (Zone 1, day 1) that was statistically significant for EDA clustering, whereas most of the conditions on Day 3 and Day 7 were statistically significant for DFA clustering. Therefore, our results demonstrate that hPSC-derived ONPs had greater tendency to cluster around DFAs than EDAs. All computed statistical values are shown in FIG. 24.

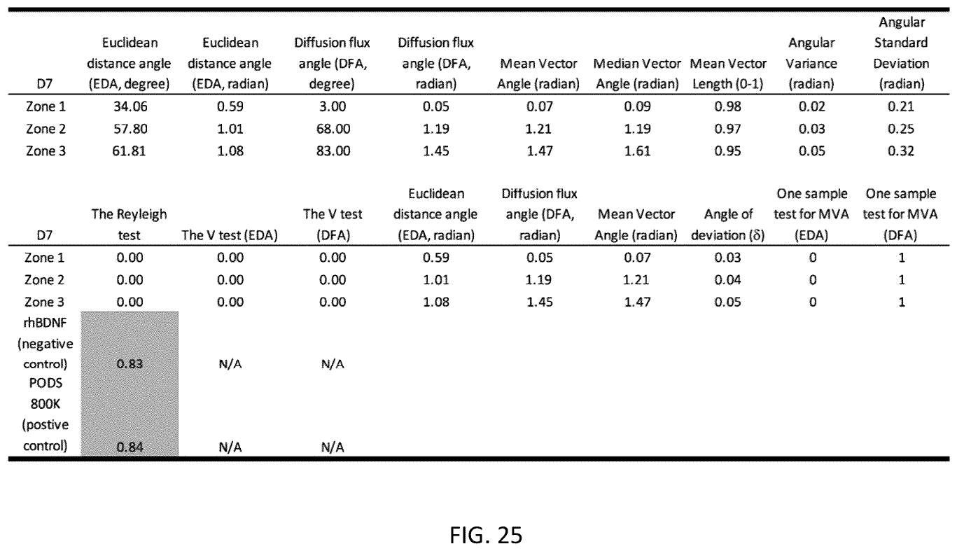

To evaluate the direction of neurites of hPSC-derived ONPs, we first defined EDA in Region 1-3 (φ′ⱼ, j=1-3) (panel A of FIG. 10) and DFA (ψ′ⱼ, j=1-3); similarly defined φᵢ and ψᵢ as in FIG. 8. All of the EDAs and DFAs defined here can be found in FIG. 25. Polar histograms of the neurite direction angle in Regions 1-3 indicate that the two longest neurites were bimodal in nature (panel B of FIG. 10). In contrast, polar histograms of those cultured with rhBDNF (negative control) and 800,000 PODS®-BNDF (positive control) did not indicate bimodal distribution the neurites did not show directionality (panel C of FIG. 10). The Rayleigh test of uniformity for both the positive and negative control were greater than 0.05, underscoring that both of the conditions had no statistically significant directionality (FIG. 25: highlighted in green). We also analyzed the direction of the neurites using circular statistics. To obtain more realistic mean vector angles, we doubled each angle and reduced the multiples modulo by 360°. In circular statistics, the bimodal distributed data can be transformed into a unimodal data by doubling the angle. The mean vector angles in panel D of FIG. 10 (right column) suggest the circumstance in which the vectors were canceled out between the two groups of angles distributed in a bimodal fashion, resulting in inaccurate representation. A circular plot in panel D of FIG. 10 (right column) showed doubled angles, representing actual representation of the neurite vector angles. In all of the three regions, the Rayleigh test and V test for EDA and DFA indicate directionality (FIG. 25). One sample test for the mean vector angles in Region 1 3 indicated that they were not statistically different from DFA, but all of the three mean vector angles were statistically different from EDA.

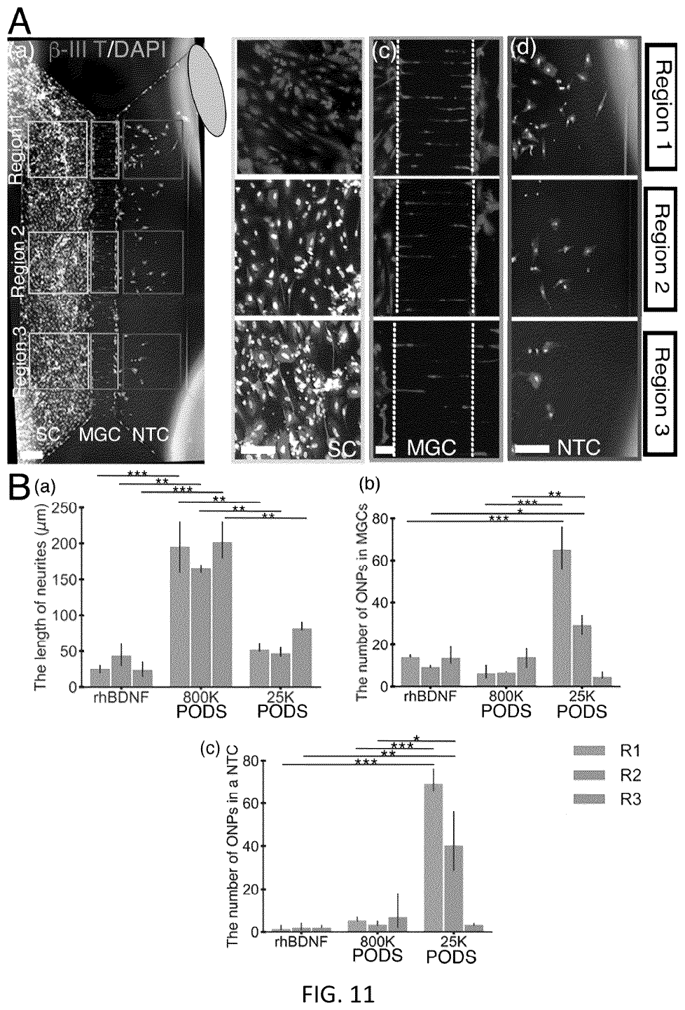

Devices were stained for β-III tubulin to track neurite growth and extension across the micro-groove channels in addition to cell migration in three selected regions (panel A of FIG. 11). Note that hPSC-derived late-stage ONPs were differentiated toward SGNs in XONA™ XC450 devices only for 7 days, resulting in a relatively immature SGN morphology (primarily bipolar with short neurites). The location of the PODS®-rhBDNF disc in relation to the regions of interest in panel A of FIG. 11 is indicated by a yellow circle. Quantitative analyses were performed and summarized in panel B of FIG. 11. Our data indicate that neurite length is dependent on rhBDNF concentration, with greater amounts of PODS®-rhBDNF crystals promoting longer neurite growth (panel B of FIG. 11, (a)). Lesser amounts of PODS®-rhBDNF, however, are necessary to create an appropriate concentration gradient. In the presence of 20,000 PODS®-rhBDNF crystals, both neurite extension into the microchannels and cell migration into the neurotrophin compartment are greatest in the region closest to the rhBDNF source and decrease further from the PODS®-rhBDNF (panel B of FIG. 11, (b,c)). Cell migration is dependent on the distance from the source of rhBDNF, thus suggesting the presence of an rhBDNF gradient as predicted by our model.

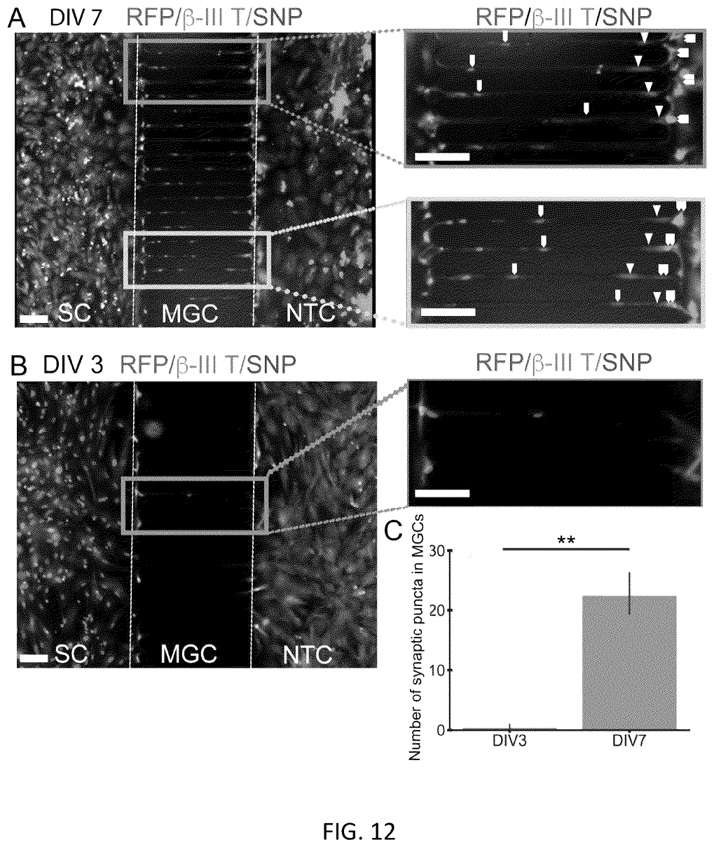

Xona™ XC450 devices were used to model the ability of transplanted hPSC-derived SGNs to form synapses with endogenous SGNs under the direction of a PODS®-induced rhBDNF concentration gradient. To generate distinguishable SGN populations in the somal and neurotrophin compartments, RFP+542 hPSC-derived late-stage ONP clones (passage number 3 after beginning early-stage ONP differentiation) were seeded into the somal compartment. hPSC-derived late-stage ONPs (passage number 5 after beginning early-stage ONP differentiation) are seeded into the neurotrophin compartment as a surrogate for endogenous SGNs. A higher passage number was used compared to cells seeded into the somal compartment to better represent the mature extant SGN population in the inner ear; higher passage number late-stage ONPs differentiate into SGNs more readily than those of lower passage numbers. As before, 20,000 PODS®-rhBDNF crystals were placed in the top right well of the neurotrophin compartment. Synapsin 1/2 expression was assessed in the microgroove channels at day in vitro 7 (DIV7) and day in vitro 3 (DIV3) to evaluate synaptic formation between cells seeded into the somal and neurotrophin compartments; our previous study demonstrated that major synaptic vesicle proteins (e.g., synapsin 1/2 and synaptophysin) were expressed at an early neuronal stage of hPSC-derived SGN development [27]. For each time-point, three technical replicates (each eld contained 10 microgroove channels×3) were performed on three biological replicates (three Xona™ XC450 devices). At DIV7, synapsin 1/2 expression was detected at the junctions between RFP+560 hPSC-derived SGN clones of the somal compartment and hPSC-derived SGNs of the neurotrophin compartment in the microgroove channels; expression of synapsin 1/2 is indicative of synaptic formation between SGNs. At DIV3, synapsin 1/2 expression was minimal at the junctions between distinct SGN populations, despite the migration of RFP+565 hPSC-derived SGN clones into the microgroove channels (panels A-B of FIG. 12). Quanti cation was performed to determine the number of synaptic puncta in the microgroove channels at DIV7 and DIV3. Student's t test indicated that the number of synaptic puncta in the microgroove channels at DIV7 was significantly greater than the number of synpatic puncta in the microgroove channels at DIV3 (p<0.01) (panel C of FIG. 12). Thus, it is apparent that hPSC-derived SGNs can create synaptic connections with other SGNs under the direction of the PODS®-rhBDNF system.

### DISCUSSION

Challenges of neurotrophin treatment in the inner ear: This proof-of-concept study for the realization of a neuroregenerative nexus was conducted to ascertain its scientific/technological parameters in a controlled in vitro environment that closely simulates the human inner ear anatomy. Neurotrophin gradients have been studied in multiple contexts. However, it has not been feasible to reliably provide, and maintain, such a gradient to neurons in vivo, primarily because of technical challenges including failure to provide a reliably sustainable source in addition to practical surgical implantation. Furthermore, while neurotrophin treatment has been recognized as a potential treatment for sensorineural hearing loss, there has not been long-term clinical success in this avenue to date. Most recent relevant clinical trials used adeno-associated virus (AAV2) to deliver BDNF to the brain. Although compelling, this treatment does not attempt to precisely control the concentration of BDNF, which could potentially interfere with normal functions in the target organ. Furthermore, this treatment may not be applicable to the inner ear, as MR-guided procedure is technically infeasible in the setting of the inner ear. In this study, we used PODS®-rhBDNF to generate and maintain a neurotrophic gradient in a controlled manner. Our results indicate that 20,000 PODS®-rhBDNF crystals allowed for an rhBDNF neurotrophin gradient such that hPSC-derived ONPs survived, differentiated toward human SGNs, and established directional neurite outgrowth in a microfludic device. Furthermore, our proposed solution has greater potential to be translated into clinical practice; in addition to its proven natural self-sustainability, we have previously demonstrated that implantation of PODS®-rhBDNF is met with little immune rejection when embedded in a nanofibrillar cellulose hydrogel in mice.

Microfluidic device-generated gradient: We utilized a microfluidic device to advance our understanding of directional neurite growth and otic neuronal differentiation in response to an rhBDNF concentration gradient. Among many in vitro concentration gradient sustaining culture devices, microfluidic devices have overcome many of the deficits that conventional platforms (i.e., the Boyden chamber, Dunn chambers, or compartmentalized diffusion chambers) face. Conventional platforms tend to be sub-optimal in manipulating small volumes of fluid at the order of microliters. Growth factors and proteins can be used in small quantities in our microfluidic device, and cultured stem cells are able to interact with endogenous factors at biologically relevant concentrations. As mentioned earlier, this micro-environment more accurately represents in vivo conditions. The Xona™ device can be used to create and sustain a three-dimensional concentration gradient over time (duration and diffusion profile dependent on the chemical kinetics of the molecule) because of its microchannel array. The device limits convective flow in the gradient-forming areas by introducing microgroove channels that generate high fluidic resistance, thereby limiting flow to diffusion. The high resistance of the microchannel array also prolongs diffusion across them, thereby increasing gradient formation and decreasing gradient steepness (essentially elongating it). These features motivated us to generate an FEM to predict the rhBDNF gradients resulting from different numbers of PODS®-rhBDNF crystals. Note, however, that this environment is geometrically different from the micro-environment in the cochlea. Therefore, a mesh geometry of the cochlea will be needed to compute the PODS®-rhBDNF crystal number for implementation in animals, as well as in further clinical translation.

Brain-derived neurotrophic factor: Over the course of the past 20-30 years, it has been established that BDNF mediates survival and differentiation activities of SGNs by binding and activating tyrosine kinase receptor kinase B (TrkB), a member of the larger family of Trk receptors. Numerous studies have reported that BDNF can palliate SGN degeneration in ototoxically deafened animals, a widely accepted model for retrograde trans-synaptic SGN degeneration secondary to hair cell destruction. Additionally, it has been confirmed that there is a positive correlation between SGN counts and CI performance. It is then safe to presume that BDNF application in CI recipients would enhance overall CI performance, by preserving extant SGNs and their neurites. Although promising, BDNF treatment in humans has not been implemented in the inner ear as simply introducing BDNF into the inner ear poses significant hurdles.

Unsuccessful BDNF treatment is attributed to several factors. The blood half-life of BDNF protein is extremely short, lasting only 1 10 minutes in plasma and one hour in CSF. Due to BDNF's high degradation rate, simple introduction of BDNF in solution would require continuous replenishment, which is impractical in the clinical setting. Furthermore, simply introducing BDNF in solution would promote non-directional neurite growth where directed neurite growth is essential, especially in the case of our proposed bioactive CI, due to the lack of a concentration gradient to guide the growth. Directing neurite growth towards the CI electrode array is pivotal in the ultimate goal of enhancing performance through the narrowing of the electrode-neuron gap. The PODS® system provides a gradual release of growth factor from a localized origin. This steady supply of BDNF from a localized origin not only generates a concentration gradient, but maintains it over time. As shown in FIG. 6, it is unmistakable that the slow-release nature of PODS®-hBDNF results in a concentration gradient over the course of Day 1-7. As shown in FIGS. 7-11, careful calibration of the resulting BDNF gradient can lead to the promotion of hPSC-derived ONP differentiation towards SGN lineage and directed neurite extension.

It should be noted that our FEM assumes free diffusion of the rhBDNF protein. In biological cell-culture conditions, rhBDNF released from PODS®-rhBDNF has tendency to adhere to walls of the culture device, due to its nature as a “sticky” protein of about 27 kDa (mature BDNF dimer) that is positively charged under physiological conditions (isoelectric point, pI=9.4). As such, the physio-chemical properties of rhBDNF have rendered the protein prone to diminished diffusive transport. This phenomenon was observed in preliminary data where the ONPs failed to survive past 1-3 days of culture (data not shown). To circumvent this issue we infused the culture media with a carrier protein (i.e., BSA), hypothesizing that the albumin would act as a carrier for the released rhBDNF and allow for free diffusive transport throughout the microfluidic device. This hypothesis is supported by our sets of biological validation data (FIGS. 7-11) clearly showing that hPSC-derived ONPs responded positively to the modification; they exhibited the expected cell characteristics, body orientation, unidirectional neurite extension, and neurite length. Note that albumin is the single most highly concentrated protein in the perilymph, therefore, an artificially introduced carrier protein would not be required in future in vivo studies.

Intracellular signaling initiated by the Tyrosine kinase B receptor: Another issue for consideration in the interpretation of our results is the intracellular cell-signaling mechanism elicited by rhBDNF. Human BDNF (mature dimeric form) binds with high affinity to the TrkB receptor. The binding of BDNF to a TrkB receptor is integral to the pro-neuronal effects of BDNF. Upon binding, TrkB dimerizes and activates intrinsic kinase activities and other complex sets of intracellular signaling cascades, beyond the scope of this study. It should be noted that activation also initiates internalization by endocytosis and subsequently transports the protein to the soma. Therefore, the pro-neuronal effects of rhBDNF in our results may be highly dependent on the status of the TrkB receptors of the cell membranes of hPSC-derived ONPs. Our previous study has demonstrated strong expression of the TrkB receptors on derived ONPs, however, more detailed studies on TrkB receptors of hPSC-derived ONPs and SGNs are needed to deepen understanding.

Degradation of PODS® crystals by protease activity: In cell culture, degradation of PODS®-rhBDNF is likely due to the enzymatic activity of cell-secreted proteases. The proteases break down the peptide bonds of the encasing polyhedrin protein, creating openings in the structure to allow release of the rhBDNF. Therefore, the presence of proteases is imperative for the proper implementation of PODS® technology. These proteases are additionally responsible for subsequent degradation of the released rhBDNF. Because cells were not present in the culture media used for the PODS® degradation kinetics experiments, we infused the media with 10% FBS, which inherently contains proteases. Furthermore, since the cells and PODS® are initially segregated into separate compartments within the culture device, cell-secreted proteases are unlikely to reach and degrade the PODS® in time to support ONP survival and differentiation, which was apparent in experiments conducted without the addition of FBS (data not shown). Infusion with FBS was therefore required in these experiments as well. For clinical translation, however, we presume that cell-secreted proteases will be readily present in the inner ear and therefore preclude the need for artificial supplementation.

Neurotrophin-gradient induced synaptogensis: To explore the full potential of the PODS®-rhBDNF system, we performed an experiment in which we placed RFP-transfected hPSC-derived late-stage ONPs in the somal compartment. We also placed RFP-negative hPSC-derived late-stage ONPs in the neurotrophin compartment with the 20,000 PODS®-BDNF. We demonstrated synaptic connections between these two populations of derived SGNs (FIG. 12). This additional result indicates that the PODS®-rhBDNF system and controlled concentration gradient can induce synaptogenesis between human pluripotent stem cell-derived SGNs, the novelty of which should be noted. While some groups used in vivo BDNF release by gene transfer or close-eld electro-gene transfer to provide a long-term release of BDNF to accomplish neurotrophin-guided synaptic connections, these were tested in rodents, in contrast to human pluripotent derived SGNs.

A concept design: neuro-regenerative nexus: The plateau in CI performance in treatment of sensorineural hearing loss has driven researchers to develop innovative supplementary treatment strategies to push the field past this hurdle. Our approach strives to directly address the issue at its core: the electrode-neuron gap which can lead to serious implications including low spatial frequency resolution and high power consumption. The data presented here can be used as a launchpad for the neuro-regenerative nexus (NRN). The proposed NRN is a biointerface concept that integrates a long-term release source of neurotrophins (i.e., BDNF) with a bioengineered scaffold to facilitate and maintain a neurotrophic factor gradient. Embedding the source (e.g., PODS®-rhBDNF) in a biocompatible matrix ensures localization as well as a mechanism to fine-tune degradation and release kinetics. Implanted in conjunction with the CI, the NRN acts as a supportive bridge between extant SGNs and transplanted hPSC-derived late-stage ONPs, which are localized on the implant electrodes themselves (see FIG. 1 for schematic). The NRN would promote survival of both cell populations, differentiation of the hPSC-derived late-stage ONP transplants towards SGNs, directional neurite growth, and synaptogenesis between the two cell populations, effectively creating a neuronal network between the patient and the CI. As a result, each electrode would stimulate cell bodies at exceptionally high resolution, essential for greater tonal differentiability (required for effective social interaction and music appreciation) and furthermore, increased quality of life for millions. The successful outcomes presented herein are pivotal for the realization of a neuroregenerative nexus that is effective in the in vivo environment.

Many groups have demonstrated that concurrent BDNF treatment, including via osmotic pumps and BDNF-producing transfected cell lines, improves the survival of SGNs. One group applied a biocompatible and electrically conductive coating immersed with BDNF to a CI electrode array. None of these techniques have a reliable and practical method for continuous supply; they provide only short-term BDNF exposure. While some have shown that eventual BDNF depletion leads to an accelerated decline in neuronal survival, other groups have shown that abrupt cessation of BDNF treatment does not lead to SGN degeneration and that temporary treatment is sufficient for their long-term survival. These data contradict, so the verdict is unclear. The integrity and function of the transplanted hPSC-derived SGNs could potentially start deteriorating after the depletion of PODS®-rhBDNF. Having said that, our biohybrid CI employs the use of electrical stimulation (ES) to the native SGNs in conjunction with transplantation of hPSC-derived SGNs to bridge the electrode-neuron gap. It has been established that ES delivered by a CI provides trophic inputs to the SGNs. These studies suggested that ES can preserve SGNs following various types of insults. The most recent study by Scheper et al. examined the functional and neuroanatomical effects of continuous BDNF treatment with consecutive chronic ES on the deafened guinea pig cochlea. The results demonstrated that chronic ES starting after cessation of the BDNF treatment could maintain increased SGN density compared to the untreated contralateral side as effectively as simultaneous combined treatment, which has been repeatably shown to be successful. Therefore, it is reasonable to hypothesize that transplanted hPSC-derived SGNs will likely maintain their integrity and function even after eventual BDNF treatment cessation. Furthermore, a recent study demonstrated that ES to the SGNs did not cause stimulus-induced degeneration if the stimulus parameters were within a standard range.

The limitations of this study and future direction: The reduction of the spacial dimension to 2D for flux analysis certainly affected the flux vector, which determines the predicted concentration vector. Given that the thickness (i.e., Z-axis) of the microfluidic device was 100 μm, estimated that the effect was minimal. In the future, we plan to use hPSC-derived 3D spheroids in the somal compartment so that flux vector and concentration gradient vector can more accurately model cell behavior. In this way, we will be able to circumvent the need to reduce diffusion calculations to 2D for computation performance in the modeling.

We generated an FEM that modeled rhBDNF's biological transport phenomenon from a PODS®-rhBDNF disc throughout the Xona™ device. Note that in this model, we focused on the major dependent variable, the concentration and diffusion profile of rhBDNF, to model the biological phenomenon. Other physical variables that promote cell migration, otic neuronal differentiation, and neurite growth were not taken into consideration. These variables include electrotaxis (electrical potential), durotaxis (matrix stiffness, i.e., laminin 511 in our case), mechanotaxis (cell strain), and lastly cell migration by random walk. In future studies, we will take these variables into consideration to more accurately represent the migration and neurite growth of hPSC-derived ONPs.

Insufficient contrast between cells and background in phase contrast images led to inaccuracies in cell orientation computation for some images. To address this issue, poor quality images were disregarded in the quantitative analysis. We occasionally used manual measurement for accuracy. Our future study may entail automated time-series cell analysis, which would allow more accurate measurement. Another alternative to address this issue would be with cell membrane staining in the future.

While 20,000 PODS®-rhBDNF crystals were necessary for otic neuronal differentiation and directional neurite outgrowth of hPSC-derived ONPs, this condition may not be sufficient. For instance, it is still not understood whether the effects of other neurotrophic factors such as Neurotrophin-3 (NT-3) and Glial cell line-derived neurotrophic factor could have a positive effect on ONP growth. We plan to investigate the effects of these and other neurotrophic factors in future studies. Other aspects that could impact directional neurite growth include endogenous factors secreted from hP SC-derived ONPs. While our previous study demonstrated that hPSC-derived ONPs only secreted negligible amounts of BDNF, quantified by ELISA, we currently lack data on other neurotrophins and molecules that can affect directional neurite growth in the inner ear. Additionally, previous studies indicate that neurotrophic support of SGNs is mainly composed of BDNF and neurotrophin-3 (NT-3). Therefore, the potential confounding effects of other neurotrophic factors (i.e., NT-3) and molecules secreted from hPSC-derived SGNs require further investigation.

The microfluidic device we used can simulate the complex anatomical environment of the inner ear. Be that as it may, there is no such guarantee for precise and controlled neurite outgrowth in the in vivo setting. To accomplish this goal, we need to perform an FEM simulation using the geometric specifications of the human cochlea and scala tympani. This will most likely lead to adaptations to the PODS® volumetric geometry and concentration distribution. It will also require a new set of boundary conditions in which BDNF will be diffused in a larger volume (the volume of the human scala tympani is 32 L), likely requiring a larger number of PODS®-BDNF crystals to generate the appropriate BDNF gradient in the scala tympani. In this study, we reconstituted the PODS® crystals in 2 L of saline. Considering the volume of the human scala tympani, it would be still feasible to place required PODS® crystals in the scala tympani even though the required number is greater than 20,000. Furthermore, we will take the presence of the helicotrema into consideration as it generates a pressure gradient between the scala tympani and scala vestibuli, which will affect the concentration gradient of PODS®-BDNF.

To implant and keep PODS®-crystals in place in the scala tympani, we propose a strip or rod incorporated with PODS®-crystals to be placed in conjunction with the implant to facilitate neural integration between the CI and transplanted SGNs as well as guide neurite outgrowth from native SGNs through the CPS. Aliphatic polyesters can be considered as representatives of synthetic biodegradable thermoplastic polymers. Among these, copolymers of Poly(lacticacid) (PLA) and Poly(glycolic acid)-Poly(lactide-co-glycolide) (PLAGA) have been extensively investigated for various medical applications such as sutures, bone pins, stents, drug delivery devices, and scaffolds for tissue engineering. The mechanical properties and degradation rate can be tuned by varying the amount of each monomer type. As a more durable alternative to a totally encapsulating (soft) hydrogel, where incorporation of PODS® is a doddle, we propose to use a biodegradable PLAGA rod inside of which contains PODS®-rhBDNF crystals. We could also incorporate a CI coated with Poly-D-Lysine/laminin with hPSC-derived SGNs. These two components would constitute the bioactive CI, held together by a biocompatible hydrogel and externally coated with a PLA/PLAGA copolymer. In this way, we have all of the components required for a bioactive CI within this apparatus, which should be readily transplanted into the scala tympani.

One of the potential hurdles in the clinical application of the neuro-regenerative nexus in the biohybrid CI is the potential for scar tissue encapsulation surrounding the electrode, leaving virtually no space for other cells to thrive. The tissue response in these studies was described as a form of a mature foreign body response composed of a compact tissue capsule, typically including a zone of macrophages around the electrode array. In addition, a loose vascularized brous tissue response was evident, occupying at least part of the scala tympani proximal to the electrode array. To circumvent this phenomenon, we propose pretreating the scala tympani with a thin layer of mesenchymal stem cells to address the issue. It is known that hMSCs are essential trophic mediators and can be readily derived from various autologous or allogeneic adult tissues. hMSCs may recapitulate several mechanisms sufficient for inhibiting scar formation in different clinical settings. Preclinical and clinical trials are in progress. Therefore, it seems feasible to incorporate this treatment in our biohybrid CI.

### CONCLUSIONS

We successfully promoted survival, neuronal differentiation toward SGNs, and directed neurite extension of hPSC-derived late-stage ONPs, and synaptogenesis between two hPSC-derived SGN cell populations by generating a neurotrophin gradient. This technique can allow for controlled neurite growth in transplanted hPSC-derived ONPs, as well as native, extant SGNs in the inner ear. This proof-of-concept study translates to a step toward next-generation bioacitve CI technology, in which the stem-cell derived neurons are incorporated upon the cochlear implant electrode strip, as the technique will allow us to employ directional neurite growth from the transplanted cells in the inner ear. This study is the first to present the concept of a “neuro-regenerative nexus” congruent with a bioactive cochlear implant to eliminate the electrode-neuron gap—the most significant barrier to next-generation cochlear implant technology.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. J. G. Naples, M. J. Ruckenstein, Cochlear Implant., Otolaryngologic Clinics of North America 53 (1) (2020) 87 102. doi:10.1016/j.otc.2019.09.004.
[2]. B. S. Wilson, M. F. Dorman, Cochlear implants: A remarkable past and a brilliant future, Hearing Research 242 (1-2) (2008) 3 21. arXiv: NI11143150003, doi: 10.1016/j. heares.2008.06.005.
[3]. C. Frick, M. Mfiller, U. Wank, A. Tropitzsch, B. Kramer, P. Senn, H. Rask-Anderson, K. H. Wiesmiiller, H. Lewenheim, Biofunctionalized peptide-based hydrogels provide permissive scaffolds to attract neurite outgrowth from spiral ganglion neurons, Colloids and Surfaces B: Biointerfaces 149 (2017) 105 114. doi: 10.1016/j colsurfb.2016.10.003.
[4]. S. Hahnewald, A. Tscherter, E. Marconi, J. Streit, H. R. Widmer, C. Gar-nham, H. Benav, M. Mueller, H. L6wenheim, M. Roccio, P. Senn, Response profiles of murine spiral ganglion neurons on multi-electrode arrays., Journal of neural engineering 13 (1) (2016) 16011. doi: 10.1088/1741-2560/13/1/016011.
[5]. R. K. Shepherd, S. Hatsushika, G. M. Clark, Electrical stimulation of the auditory nerve: The effect of electrode position on neural excitation, Hearing Research 66 (1) (1993) 108 120. doi:10.1016/0378-5955 (93) 90265-3.
[6]. M. Tykocinski, L. T. Cohen, B. C. Pyman, T. Roland, C. Treaba, J. Pala-mara, M. C. Dahm, R. K. Shepherd, J. Xu, R. S. Cowan, N. L. Cohen, G. M. Clark, Comparison of electrode position in the human cochlea using various perimodiolar electrode arrays., The American journal of otology 21 (2) (2000) 205 211. doi: 10.1016/S0196-0709 (00)80010-1.
[7]. Roemer, U. Kohl, O. Majdani, S. K16B, C. Falk, S. Haumann, T. Lenarz, A. Kral, A. Warnecke, Biohybrid cochlear implants in human neurosensory restoration., Stem cell research and therapy 7 (1) (2016) 148. doi:10.1186/s13287-016-0408-y.
[8]. R. A. Heuer, K. T. Nella, H. T. Chang, K. S. Coots, A. M. Oleksijew, C. B. Roque, L. H. Silva, T. L. McGuire, K. Homma, A. J. Matsuoka, Three-dimensional otic neuronal progenitor spheroids derived from human embryonic stem cells, Tissue Engineering—Part A 27 (3-4) (2021) 256 269. doi: 10.1089/ten. tea. 2020.0078.
[9]. H.-T. Chang, R. A. Heuer, A. M. Oleksijew, K. S. Coots, C. B. Roque, K. T. Nella, T. L. McGuire, A. J. Matsuoka, An Engineered Three-Dimensional Stem Cell Niche in the Inner Ear by Applying a Nanofibrillar Cellulose Hydrogel with a Sustained-Release Neurotrophic Factor Delivery System, Acta Biomaterialia 108 (2020) 111 127. doi:https://doi.org/10.1016/j.actbio.2020.03.007.
[10]. L. Taylor, L. Jones, M. H. Tuszynski, A. Blesch, Neurotroph n-3 gradients established by lentiviral gene delivery promote short-distance axonal bridging beyond cellular grafts in the injured spinal cord, Journal of Neuroscience 26 (38) (2006) 9713 9721. doi:10.1523/JNEUROSCI.0734-06.2006.
[11]. P. Senn, M. Roccio, S. Hahnewald, C. Frick, M. Kwiatkowska, M. Ishikawa, P. Bako, H. Li, F. Edin, W. Liu, H. Rask-Andersen, I. Pyykke, J. Zou, M. Mannerstrem, H. Keppner, A. Homsy, E. Laux, M. Llera, J. P. Lel-louche, S. Ostrovsky, E. Banin, A. Gedanken, N. Perkas, U. Wank, K. H. Wiesmiiller, P. Mistrik, H. Benav, C. Garnham, C. Jolly, F. Gander, P. Ulrich, M. Muller, H. Lewenheim, NANOCI-Nanotechnology Based Cochlear Implant with Gapless Interface to Auditory Neurons, Otology and Neuro-tology 38 (8) (2017) e224-e231. doi: 10.1097/MA0.0000000000001439.
[12]. G. J. Goodhill, H. Baier, Axon Guidance: Stretching Gradients to the Limit, Neural Computation 10 (3) (1998) 521 527. doi:10.1162/089976698300017638.
[13]. L. N. Gillespie, G. M. Clark, P. F. Bartlett, P. L. Marzella, BDNF-induced survival of auditory neurons in vivo: Cessation of treatment leads to accelerated loss of survival effects, Journal of Neuroscience Research 71 (6) (2003) 785 790. doi: 10.1002/jnr.10542.
[14]. L. N. Pettingill, R. L. Minter, R. K. Shepherd, Schwann cells genetically modified to express neurotrophins promote spiral ganglion neuron survival in vitro, Neuroscience 152 (3) (2008) 821 828. doi:10.1016/j. neuroscience. 2007.11.057. Schvarat
[15]. R. K. Shepherd, A. Coco, S. B. Epp, Neurotrophins and electrical stimulation for protection and repair of spiral ganglion neurons following sensorineural hearing loss, Hearing Research 242 (2009) 100 109. doi: 10.1016/j. heares. 2007.12.005.Neurotrophins
[16]. B. Baseri, J. J. Choi, T. Deffieux, G. Samiotaki, Y. S. Tung, O. Olu-molade, S. A. Small, B. Morrison, E. E. Konofagou, Activation of signaling pathways following localized delivery of systemically administered neurotrophic factors across the bloodbrain barrier using focused ultrasound and microbubbles, Physics in Medicine and Biology 57 (7). doi: 10.1088/0031-9155/57/7/N65.
[17]. K. Ikeda, S. Nagaoka, S. Winkler, K. Kotani, H. Yagi, K. Nakanishi, S. Miyajima, J. Kobayashi, H. Mori, Molecular Characterization of Bom-byx mori Cytoplasmic Polyhedrosis Virus Genome Segment 4, Journal of Virology 75 (2) (2001) 988 995. doi: 10.1128/j vi. 75.2.988-995.2001.
[18]. T. Suzuki, T. Kanaya, H. Okazaki, K. Ogawa, A. Usami, H. Watanabe, K. Kadono-Okuda, M. Yamakawa, H. Sato, H. Mori, S. Takahashi, K. Oda, Efficient protein production using a Bombyx mori nuclear polyhedrosis virus lacking the cysteine proteinase gene, Journal of General Virology 78 (12) (1997) 3073 3080. doi: 10.1099/0022-1317-78-12-3073.
[19]. H. Mori, R. Ito, H. Nakazawa, M. Sumida, F. Matsubara, Y. Minobe, Expression of Bombyx mori cytoplasmic polyhedrosis virus polyhedrin ininsect cells by using a baculovirus expression vector, and its assembly into polyhedra, Journal of General Virology 74 (1) (1993) 99 102. doi:10. 1099/0022-1317-74-1-99.
[20]. S. H. Green, E. Bailey, Q. Wang, R. L. Davis, The Trk A, B, C's of Neurotrophins in the Cochlea., Anatomical record (Hoboken, N.J.: 2007) 295 (11) (2012) 1877 1895. doi:10.1002/ar.22587.
[21]. Meyvantsson, D. J. Beebe, Cell culture models in microfluidic systems, Annual Review of Analytical Chemistry 1 (1) (2008) 423 449. doi: 10. 1146/annurev. anthem. 1.031207.113042.
[22]. Z.-J. Guo, M.-H. Yu, X.-Y. Dong, W.-L. Wang, T. Tian, X.-Y. Yu, X.-D. Tang, Protein composition analysis of polyhedra matrix of Bombyx mori nucleopolyhedrovirus (BmNPV) showed powerful capacity of polyhedra to encapsulate foreign proteins., Scientific reports 7 (1) (2017) 8768. doi: 10.1038/s41598-017-08987-8.
[23]. U. K. Laemmli, Cleavage of structural proteins during the assembly of the head of bacteriophage T4., Nature 227 (5259) (1970) 680 685. doi: 10.1038/227680a0.
[24]. L. Mandel, H. Ozdener, V. Utermohlen, Identification of pro- and mature brain-derived neurotrophic factor in human saliva., Archives of oral biology 54 (7) (2009) 689 695. doi: 10.1016/j.archoralbio.2009.04.005.
[25]. A. Schneider, W. S. Rasband, K. W. Eliceiri, NIH Image to ImageJ: 25 years of image analysis, Nature Methods 9 (7) (2012) 671 675. arXiv: arXiv:1011.1669v3, doi: 10.103 8/nmeth. 2089.
[26]. J. Matsuoka, Z. A. Sayed, N. Stephanopoulos, E. J. Berns, A. R. Wad-hwani, Z. D. Morrissey, D. M. Chadly, S. Kobayashi, A. N. Edelbrock, T. Mashimo, C. A. Miller, T. L. McGuire, S. I. Stupp, J. A. Kessler, Creating a stem cell niche in the inner ear using self-assembling peptide amphiphiles., Plos One 12 (12) (2017) e0190150. doi: 10.1371/journal. pone.0190150.
[27]. J. Matsuolia, Z. D. Morrissey, C. Zhang, K. Homma, A. Belmadani, C. A. Miller, D. M. Chadly, S. Kobayashi, A. N. Edelbrock, M. Tanaka-Matakatsu, D. S. Whitton, L. Lyass, T. L. McGruire, S. I. Stupp, J. A. Kessler, Directed Differentiation of Human Embryonic Stem Cells Toward Placode-Derived Spiral Ganglion-Like Sensory Neurons, Stem Cells Translational Medicine 6 (2017) 923 936. doi: 10.1002/sctm. 16-0032.
[28]. H. Al-Ali, S. R. Beckerman, J. L. Bixby, V. P. Lemmon, In vitro models of axon regeneration, Experimental Neurology 287 (Pt 3) (2017) 423 434. doi: 10.1016/j expneurol 20/6.01.020.
[29]. M. Tuncel, H. S. SUB:len, K. M. Erbil, A. Konan, Formation of the cochlear nerve in the modiolus of the guinea pig and human cochleae., Archives of medical research 36 (5) (2005) 436 40. doi: 10.1016/j. arcmed. 2005.02. 003.
[30]. B. Kiffirik, K. Abe, T. Ushiki, Y. Inuyama, S. Fukuda, Kazuo Ishikawa, Microstructures of the Bony Modiolus in the Human Cochlea: A Scanning Electron Microscopic Study, Journal of Electron Microsc 40 (40) (1991) 193 197.
[31]. R. W. Burry, Controls for immunocytochemistry: an update., The journal of histochemistry and cytochemistry: official journal of the Histochemistry Society 59 (1) (2011) 6 12. doi: 10.1369/jhc.2010.956920.
[32]. M. B. Mazalan, M. A. B. Ramlan, J. H. Shin, T. Ohashi, Effiect of geometric curvature on collective cell migration in tortuous microchannel devices, Micromachines 11 (7) (2020) 1 17. doi: 10.3390/MI11070659.
[33]. F. Xu, T. Beyazoglu, E. Hefner, U. A. Gurkan, U. Demirci, Automated and adaptable quantification of cellular alignment from microscopic images for tissue engineering applications, Tissue Engineering—Part C: Methods 17 (6) (2011) 641 649. doi: 10.1089/ten.tec. 2011.0038.
[34]. E. Batschelet, Circular Statistics in Biology (Mathmatics in Biology Series), 1st Edition, Academic Press, New York, NY, 1981.
[35]. P. Berens, M. J. Velasco, CircStat for Matlab: Toolbox for circular statistics with Matlab, Max Planck Institut fur biologische Kybernetik, Technical Report No. 184 31 (10) (2009) 1 21. doi:doi:10.18637/j ss.v031.110.
[36]. M. Pool, J. Thiemann, A. Bar-Or, A. E. Fournier, NeuriteTracer: A novel ImageJ plugin for automated quantification of neurite outgrowth, Journal of Neuroscience Methods 168 (1) (2008) 134 139. doi:10.1016/j.jneumeth.2007.08.029.
[37]. E. Meijering, M. Jacob, J.-C. F. Sarria, P. Steiner, H. Hirling, M. Unser, Design and validation of a tool for neurite tracing and analysis in fluorescence microscopy images., Cytometry. Part A: the journal of the International Society for Analytical Cytology 58 (2) (2004) 167 176. doi: 10.1002/cyto. a. 20022.
[38]. M. Anniko, W. Arnold, T. Stigbrand, A. Strom, The Human Spiral Ganglion, ORL 57 (1995) 68 77.
[39]. S. van der Walt, S. C. Colbert, G. Varoquaux, The NumPy Array: A Structure for Efficient Numerical Computation, Computing in Science Engineering 13 (2) (2011) 22 30. doi: 10.1109/MCSE. 2011.37.
[40]. J. D. Hunter, Matplotlib: A 2D Graphics Environment, Computing in Science Engineering 9 (3) (2007) 90 95. doi: 10.1109/MCSE. 2007.55.
[41]. P. Virtanen, R. Gommers, T. E. Oliphant, M. Haberland, T. Reddy, D. Cournapeau, E. Burovski, P. Peterson, W. Weckesser, J. Bright, S. J. van der Walt, M. Brett, J. Wilson, K. J. Millman, N. Mayorov, A. R. J. Nelson, E. Jones, R. Kern, E. Larson, C. J. Carey, I. Polat, Y. Feng, E. W. Moore, J. VanderPlas, D. Laxalde, J. Perktold, R. Cimrman, I. Henriksen, E. A. Quintero, C. R. Harris, A. M. Archibald, A. H. Ribeiro, Pedregosa, P. van Mulbregt, SciPy 1.0: fundamental algorithms for scientific computing in Python., Nature methods 17 (3) (2020) 261 272. doi:10.1038/s41592-019-0686-2.
[42]. P. Berens, CircStat: A MATLAB Toolbox for Circular Statistics, Journal of Statistical Software 31 (10). doi: 10.18637/j ss v031. i10.
[43]. Levenspiel, Chemical Reaction Engineering, 3rd Edition, John Wiley and Sons, Inc., New York, NY, 1999.
[44]. M. Stroh, W. R. Zipfel, R. M. Williams, S. C. Ma, W. W. Webb, W. M. Saltzman, Multiphoton microscopy guides neurotrophin modification with poly(ethylene glycol) to enhance interstitial diffusion, Nature Materials 3 (7) (2004) 489 494. doi:10.1038/nmat1159.
[45]. H. Rask-Andersen, W. Liu, E. Erixon, A. Kinnefors, K. Pfaller, A. Schrott-Fischer, R. Glueckert, Human cochlea: Anatomical characteristics and their relevance for cochlear implantation, Anatomical Record 295 (11) (2012) 1791 1811. doi: 10.1002/ar. 22599.
[46]. S. Axler, Linear Algebra Done Right, 3rd Edition, Springer Publishing, New York, NY, 2015.
[47]. J. Crank, The mathmatics of Diffusion, 2nd Edition, Oxford University Press, London, UK, 1979.
[48]. K. M. Keefe, I. S. Sheikh, G. M. Smith, Targeting Neurotrophins to Specific Populations of Neurons: NGF, BDNF, and NT-3 and Their Relevance for Treatment of Spinal Cord Injury., International journal of molecular sciences 18 (3). doi:10.3390/ijms18030548.
[49]. B. I. Awad, M. A. Carmody, M. P. Steinmetz, Potential role of growth factors in the management of spinal cord injury., World Neurosurgery 83 (1) (2015) 120 131. doi: 10.1016/j.wneu. 2013.01.042.
[50]. E. R. n. Hollis, M. H. Tuszynski, Neurotrophins: potential therapeutic tools for the treatment of spinal cord injury., Neurotherapeutics: the journal of the American Society for Experimental NeuroTherapeutics 8 (4) (2011) 694 703. doi:10.1007/s13311-011-0074-9.
[51]. H. Nagahara, B. R. Wilson, I. Ivasyk, I. Kovacs, S. Rawalji, J. R. Bringas, P. J. Pivirotto, W. S. Sebastian, L. Samaranch, K. S. Bankiewicz, M. H. Tuszynski, MR-guided delivery of AAV2-BDNF into the entorhi-nal cortex of non-human primates., Gene therapy 25 (2) (2018) 104 114. doi:10.1038/s41434-018-0010-2.
[52]. S. D. Croll, C. Suri, D. L. Compton, M. V. Simmons, G. D. Yan-copoulos, R. M. Lindsay, S. J. Wiegand, J. S. Rudge, H. E. Scharfman, Brain-derived neurotrophic factor transgenic mice exhibit passive avoidance deficits, increased seizure severity and in vitro hyperexcitability in the hippocampus and entorhinal cortex., Neuroscience 93 (4) (1999) 1491 1506. doi: 10.1016/50306-4522 (99) 00296-1.
[53]. Dravid, S. Parittotokkaporn, Z. Aqrawe, S. J. O'Carroll, D. Svirskis, Determining Neurotrophin Gradients in Vitro to Direct Axonal Outgrowth following Spinal Cord Injury, ACS Chemical Neuroscience 11 (2) (2020) 121 132. doi:10.1021/acschemneuro.9b00565.
[54]. T. Yamagata, J. M. Miller, M. Ulfendahl, N. P. Olivius, R. A. Altschuler, I. Pyykko, G. Bredberg, Delayed neurotrophic treatment preserves nerve survival and electrophysiological responsiveness in neomycin-deafened guinea pigs., Journal of neuroscience research 78 (1) (2004) 75 86. doi: 10.1002/jnr.20239.
[55]. M. P. Zanin, M. Hellstr??m, R. K. Shepherd, A. R. Harvey, L. N. Gillespie, Development of a cell-based treatment for long-term neurotrophin expression and spiral ganglion neuron survival, Neuroscience 277 (2014) 690 699. doi: 10.1016/j. neuroscience.2014.07.044.
[56]. M. Seyyedi, L. Viana, J. J. Nadol, Within-subject comparison of word recognition and spiral ganglion cell count in bilateral cochlear implant recipients, Otol Neurotol 35 (8) (2014) 1446 1450. doi:10.1097/MAO. 0000000000000443.Within-Subject.
[57]. Henriques, C. Pitzer, A. Schneider, Neurotrophic growth factors for the treatment of amyotrophic lateral sclerosis: where do we stand?, Frontiers in neuroscience 4 (2010) 32. doi:10.3389/fnins.2010.00032.
[58]. J. F. Poduslo, G. L. Curran, Permeability at the blood-brain and blood-nerve barriers of the neurotrophic factors: NGF, CNTF, NT-3, BDNF, Molecular Brain Research 36 (2) (1996) 280 286. doi:10.1016/0169-328X (95) 00250-V.
[59]. T. Sakane, W. M. Pardridge, Carboxyl-directed pegylation of brain-derived neurotrophic factor markedly reduces systemic clearance with minimal loss of biologic activity (1997). doi:10.1023/A:1012117815460.
[60]. R. G. Soderquist, E. D. Milligan, E. M. Sloane, J. A. Harrison, K. K. Douvas, J. M. Potter, T. S. Hughes, R. A. Chavez, K. Johnson, L. R. Watkins, M. J. Mahoney, PEGylation of brain-derived neurotrophic factor for preserved biological activity and enhanced spinal cord distribution., Journal of biomedical materials research. Part A 91 (3) (2009) 719 729. doi: 10.1002/j bm. a. 32254.
[61]. M. Sasi, B. Vignoli, M. Canossa, R. Blum, Neurobiology of local and intercellular BDNF signaling, Pflugers Archiv: European journal of physiology 469 (5-6) (2017) 593 610. doi:10.1007/200424-017-1964-4.
[62]. X. Li, Y. Su, S. Liu, L. Tan, X. Mo, S. Ramakrishna, Encapsulation of proteins in poly(l-lactide-co-caprolactone) fibers by emulsion electro-spinning, Colloids and Surfaces B: Biointerfaces 75 (2) (2010) 418 424. doi: 10.1016/j colsurfb 2009.09.014.
[63]. E. E. L. Swan, M. Peppi, Z. Chen, K. M. Green, J. E. Evans, M. J. McKenna, M. J. Mescher, S. G. Kujawa, W. F. Sewell, Proteomics analysis of perilymph and cerebrospinal fluid in mouse The Laryngoscope 119 (5) (2009) 953 958. doi: 10.1002/lary. 20209.
[64]. T. Numakawa, S. Suzuki, E. Kumamaru, N. Adachi, M. Richards, H. Kunugi, BDNF function and intracellular signaling in neurons, Histology and Histopathology 25 (2) (2010) 237 258. doi:10.14670/HH-25.237.
[65]. C. Gentile, Engineering of Spheroids for Stem Cell Technology, Current Stem Cell Research & Therapy 11 (2016) 652 665. doi:10.2174/1574888x10666151001114848.
[66]. H. Berg, Random Walks in Biology, Princeton Universtiy Press, Prinston, N J, 1983.
[67]. H. Li, F. Edin, H. Hayashi, O. Gudjonsson, N. Danckwardt-Lillies, H. En-gqvist, H. Rask-Andersen, W. Xia, Guided growth of auditory neurons: Bioactive particles towards gapless neural electrode interface, Biomaterials 122 (2017) 1 9. doi: 10.1016/j. biomaterials.2016.12.020.
[68]. J. Schulze, H. Staecker, D. Wedekind, T. Lenarz, A. Warnecke, Expression pattern of brain-derived neurotrophic factor and its associated receptors: Implications for exogenous neurotrophin application., Hearing Research 413 (2020) 108098. doi: 10.1016/j.heares. 2020.108098.
[69]. J. Matsuoka, Z. D. Morrissey, C. Zhang, K. Homma, A. Belmadani, C. A. Miller, D. M. Chadly, S. Kobayashi, A. N. Edelbrock, M. Tanaka-Matakatsu, D. S. Whitlon, L. Lyass, T. L. McGruire, S. I. Stupp, J. A. Kessler, Directed Differentiation of Human Embryonic Stem Cells Toward Placode-Derived Spiral Ganglion-Like Sensory Neurons, Stem Cells Translational Medicine 6 (2017) 923-936. doi:10.1002/sctm.16-0032.
[70]. Burgess, S. Vigneron, E. Brioudes, J.-C. Labbe, T. Lorca, A. Castro, Loss of human Greatwall results in G2 arrest and multiple mitotic defects due to deregulation of the cyclin B-Cdc2/PP2A balance., Proceedings of the National Academy of Sciences of the United States of America 107 (28) (2010) 1256412569. doi:10.1073/pnas.0914191107.
[71]. Gavet, J. Pines, Europe PMC Funders Group Progressive activation of CyclinB 1-Cdkl coordinates entry to mitosis 18 (4) (2012) 533-543. doi: 10.1016/j.devce1.2010.02.013.Progressive.
[72]. J. Byun, M. R. Verardo, B. Sumengen, G. P. Lewis, B. S. Manjunath, S. K. Fisher, Automated tool for the detection of cell nuclei in digital microscopic images: Application to retinal images, Molecular vision 12 (August) (2006) 949-960.
[73]. E. Xu, T. Beyazoglu, E. Hefner, U. A. Gurkan, U. Demirci, Automated and adaptable quantification of cellular alignment from microscopic images for tissue engineering applications, Tissue Engineering—Part C: Methods 17 (6) (2011) 641-649. doi: 10.1089/ten.tec.2011.0038.
[74]. Christos Loizou, Image Despeckle Filtering Toolbox, (2021). https://www.mathworks.com/matlabcentral/fileexchange/54044-image-despeckle-filtering-toolbox.
[75]. Leonardo O. Theme, Gaussian Bandpass Filter for Image Processing (2021). https://www.mathworks.com/matlabcentral/fileexchange/30947-gaussian-bandpass-filter-for-image-processing.
[76]. J. Sauvola and M. Pietikainen, Adaptive document image binarization, Pattern Recognition 33 (2) (2000) 225-236.
[77]. Jan Motl, Sauvola local image thresholding (2021). https://www.mathworks.com/matlabcentral/fileexchange/40266-sauvola-local-image-thresholding.
[78]. R Soille, Morphological Image Analysis: Principles and Applications., Springer-Verlag, Berlin, Germany, 1999.
[79]. F. Carricondo, B. Romero-Gomez, The Cochlear Spiral Ganglion Neurons: The Auditory Portion of the VIII Nerve., Anatomical record (Hoboken, N.J.: 2007) 302 (3) (2019) 463-471. doi: 10.1002/ar.23815.
[80]. M. Arendse, Magnetic field detection in diestinct from light detection in the invertebrates Tenebrio an Talitrus., Nature 274 (July) (1978) 358-362.
[81]. R Berens, CircStat: A MATLAB Toolbox for Circular Statistics, Journal of Statistical Software 31 (10). doi:10.18637/j ss.v031. i10.
[82]. J. H. Zar, Biostatistical Analysis, 5th Edition, Upper Saddle River, NJ, U.S.A., 2010.
[83]. L. Perrin, Linear or Nonlinear Least-Squares Analysis of Kinetic Data?, Journal of Chemical Education 94 (6) (2017) 669-672. do: 10.102 l/acs. j chemed. 6b00629.
[84]. N. I. Fisher, Statistical Analysis of Circular Data, first edit Edition, Cambridge, UK, 1993.
[85]. Pewsey, M. Neuhauser, G. D. Ruxton, Circular Statistics in R, Oxford University Press, Oxford, U K, 2013.
[86]. Albert Edge, Stefan Heller, Cochlear implants containing biological cells and uses thereof, U.S. Patent Application Publication No. 20070093878, Apr. 26, 2007.
[87]. Yuka Matsuzaki, Hajime Mori, Christian Pernstich, Michael Howard Jones, Delivery method, U.S. Patent Application Publication No.: 20200277570, Sep. 3, 2020.
[88]. Huawei Li, Albert Edge, Stefan Heller, Use of stem cells to generate inner ear cells, U.S. Patent Application Publication No.: 20080267929, Oct. 30, 2008.

What is claimed is:

1. A bioactive implant to be implanted into a target region of a subject, comprising:
an electrode array; and
a source of neurotrophins coupled with the electrode array for generating a neurotrophin concentration gradient that facilitates a neuro-regenerative nexus (NRN) that enables survival, neuronal differentiation toward spiral ganglion neurons (SGNs), and directed neurite extension of human pluripotent stem cell (hPSC)-derived SGNs.

2. The bioactive implant of claim 1, wherein the neurotrophin concentration gradient confers directional neurite growth from transplanted cells in the target region of the subject.

3. The bioactive implant of claim 1, wherein the neurotrophin concentration gradient comprises a brain-derived neurotrophic factor (BDNF) concentration gradient.

4. The bioactive implant of claim 1, wherein the NRN is a biological interface that doubly preserves endogenous SGNs while precisely directing growth of neurites arising from transplanted hPSC-derived otic neuronal progenitors (ONPs) toward endogenous SGNs, and vice versa.

5. The bioactive implant of claim 4, wherein the NRN acts as a supportive bridge between extant SGNs and transplanted hPSC-derived SGNs that are localized on the electrode array.

6. The bioactive implant of claim 4, wherein the NRN stimulates directed neurite outgrowth from both the hPSC-derived ONPs and the endogenous SGNs via the neurotrophic factor gradient.

7. The bioactive implant of claim 6, wherein the neurotrophic factor gradient promotes directed neurite growth of the hPSC-derived SGNs and induce synaptogenesis between two such cell populations.

8. The bioactive implant of claim 4, wherein the NRN integrates the source of neurotrophins with the electrode array to facilitate and maintain the neurotrophic factor gradient.

9. The bioactive implant of claim 1, wherein a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

10. The bioactive implant of claim 9, wherein the polyhedrin delivery system comprises a crystalline growth factor formulation to facilitate an extended release of growth factors including neurotrophins.

11. The bioactive implant of claim 10, wherein the polyhedrin delivery system is configured to encase the growth factors into polyhedrin protein crystals to produce growth factor co-crystals that have slow degradation profiles under physiological conditions, thereby allowing the extended release of the embedded bioactive growth factors.

12. The bioactive implant of claim 11, wherein the polyhedrin delivery system contains polyhedrin protein and cargo protein co-expressed within the polyhedrin crystal, wherein the cargo protein comprises rhBDNF and is controllably releasable.

13. The bioactive implant of claim 9, wherein the source of neurotrophins comprises recombinant human brain-derived neurotrophic factors (rhBDNF).

14. The bioactive implant of claim 9, wherein coupling of the polyhedrin delivery system with the electrode array establishes a neuronal network between transplanted hPSC-derived ONP grafts and extant SGNs in the target region of the subject.

15. The bioactive implant of claim 14, wherein the establishment of the neural network results in lower electrical impedance and current requirements of the bioactive implant.

16. The bioactive implant of claim 1, wherein the neuro-regenerative nexus congruent with the bioactive implant eliminates an electrode-neuron gap.

17. The bioactive implant of claim 1, being a cochlear implant (CI) implanted into an inner ear of the subject.

18. The bioactive implant of claim 1, wherein the electrode array is coated with Poly-D-Lysine/laminin with hPSC-derived SGNs.

19. The bioactive implant of claim 1, wherein the source of neurotrophins is incorporated into a strip or rod that is placed in conjunction with the electrode array to facilitate neural integration between the electrode array and transplanted SGNs and guide neurite outgrowth from native SGNs.

20. The bioactive implant of claim 19, wherein the strip or rod is formed of a biodegradable and biocompatible thermoplastic polymer, and/or a biocompatible hydrogel.

21. A method for realization of a neuro-regenerative nexus (NRN) in a target region of a subject, comprising:
placing an bioactive implant into the target region, wherein the bioactive implant comprises an electrode array and a source of neurotrophins; and
coupling the source of neurotrophins with the electrode array to generate a neurotrophin concentration gradient that facilitates the NRN for survival, neuronal differentiation toward spiral ganglion neurons (SGNs), and directed neurite extension of human pluripotent stem cell (hPSC)-derived SGNs.

22. The method of claim 21, wherein the neurotrophin concentration gradient confers directional neurite growth from the transplanted cells in the target region of the subject.

23. The method of claim 21, wherein the neurotrophin concentration gradient comprises a brain-derived neurotrophic factor (BDNF) concentration gradient.

24. The method of claim 21, wherein the NRN is a biological interface that doubly preserves endogenous SGNs while precisely directing the growth of neurites arising from transplanted hPSC-derived otic neuronal progenitors (ONPs) toward endogenous SGNs, and vice versa.

25. The method of claim 25, wherein the NRN acts as a supportive bridge between extant SGNs and transplanted hPSC-derived SGNs that are localized on the electrode array.

26. The method of claim 25, wherein the NRN stimulates directed neurite outgrowth from both hPSC-derived ONPs and endogenous SGNs via the neurotrophic factor gradient.

27. The method of claim 21, wherein a polyhedrin delivery system is adapted as the source of neurotrophins for stably providing and maintaining the neurotrophin concentration gradient to hPSC-derived ONPs, thereby facilitating otic neuronal differentiation and directional neurite outgrowth.

28. The method of claim 27, wherein the polyhedrin delivery system is configured to encase the growth factors into polyhedrin protein crystals to produce growth factor co-crystals that have slow degradation profiles under physiological conditions, thereby allowing the extended release of the embedded bioactive growth factors.

29. The method of claim 28, wherein the polyhedrin delivery system contains polyhedrin protein and cargo protein co-expressed within the polyhedrin crystal, wherein the cargo protein comprises rhBDNF and is controllably releasable.

30. The method of claim 27, wherein the source of neurotrophins comprises recombinant human brain-derived neurotrophic factors (rhBDNF).

31. The method of claim 27, wherein said coupling of the polyhedrin delivery system with the electrode array establishes a neuronal network between transplanted hPSC-derived ONP grafts and extant SGNs in the target region of the subject.

32. The method of claim 31, wherein said coupling of the polyhedrin delivery system with the electrode array comprises
applying current to the electrode array to generate electrical stimulation to the target region; and
releasing the embedded bioactive growth factors into the target region.

33. The method of claim 21, wherein the bioactive implant is a cochlear implant (CI) implanted into an inner ear of the subject.

34. The method of claim 21, wherein the electrode array is coated with Poly-D-Lysine/laminin with hPSC-derived SGNs.

35. The method of claim 21, wherein the source of neurotrophins is incorporated into a strip or rod that is placed in conjunction with the electrode array to facilitate neural integration between the electrode array and transplanted SGNs and guide neurite outgrowth from native SGNs.
